(19) United States
(12) Patent Application Publication
(10) Pub. No.: US 2022/0010355 A1
(43) Pub. Date: Jan. 13, 2022

(54) CELL BASED METHOD FOR DETERMINATION OF BOTULINUM TOXIN POTENCY BASED ON WESTERN BLOTTING

(71) Applicant: Galderma Holding SA, La Tour-de-Peilz (CH)

(72) Inventors: Emilia LEKHOLM, Uppsala (SE); Sara JACOBSON, Uppsala (SE); Sofie HELLSTEN, Uppsala (SE); Robert FREDRIKSSON, Uppsala (SE); Anh-Tri DO, Uppsala (SE)

(73) Assignee: Galderma Holding SA, La Tour-de-Peilz (CH)

(21) Appl. No.: 17/372,236

(22) Filed: Jul. 9, 2021

Related U.S. Application Data
(60) Provisional application No. 63/050,461, filed on Jul. 10, 2020.

(51) Int. Cl. C12Q 1/37

(52) U.S. Cl. CPC C12Q 1/37; G01N 2333/952

(57) ABSTRACT

Described are cell-based methods for detecting botulinum neurotoxin (BoNT) potency and/or activity in the absence of LD₅₀ assays that rely upon large numbers of laboratory animals.

### CROSS-REFERENCE TO RELATED APPLICATIONS

This application claims the benefit of priority to U.S. Application No. 63/050,461, filed Jul. 10, 2020, which is herein incorporated by reference in its entirety.

The present disclosure relates to the field of botulinum toxin potency screening for preparing compositions comprising botulinum toxins.

### BACKGROUND

The botulinum neurotoxins (BoNTs) are a family of structurally similar, but antigenically distinct protein neurotoxins which act on the peripheral nervous system to block neuromuscular transmission. These neurotoxins are extremely potent, and with a human lethal dose in the order of micrograms, give rise to the rare but frequently fatal disease, botulism. Assays for the botulinum neurotoxins are currently used in both the food and pharmaceutical industry. The food industry employs assays for the botulinum neurotoxins to validate new food packaging methods and to ensure food safety. With the growing clinical use of the botulinum toxins, the pharmaceutical industry requires accurate assays for these toxins for both product formulation and quality control.

It is known to assay for botulinum toxin in foodstuffs using the mouse lethality test. This test has been the industry standard for many years, though over the past 10 years a number of immunoassay methods have been developed in an attempt to replace the mouse test in the majority of applications.

One such assay operates by addition of a test sample to a plate or column to which is attached an antibody that binds to toxin present in the sample. A further antibody is typically used to detect bound toxin. These enzyme-linked immunoassays (ELISAs) have the advantages that they are specific to one botulinum toxin type and can be performed rapidly, in less than 2 hours. The ELISAs, however, suffer from several drawbacks: (1) they do not measure the biological activity of the toxins, (2) they cannot distinguish between active and inactive toxin, and (3) due to antigenic variations, some toxins are not detected by these assays which therefore give rise to false negatives.

The botulinum neurotoxins are known to possess highly specific zinc-endopeptidase activities within their light sub-units. Depending on the neurotoxin type, these act to cleave small proteins within the nerve cell which are involved in neurotransmitter release. Botulinum types A (BoNT/A), E (BoNT/E), and C (BoNT/C) toxins cleave the protein, SNAP-25. Botulinum types B, D, F and G and tetanus toxins cleave vesicle-associated membrane protein (VAMP—also called synaptobrevin). Botulinum type C toxin cleaves the protein syntaxin.

In the development of further toxin assays, various procedures have been devised for the evaluation of endopeptidase activities. Liquid chromatography procedures are known and are based on resolution of the peptide product and subsequent evaluation. These procedures are time-consuming, expensive, and do not lend themselves readily to automation. It is also known to use spectrophotometric methods, requiring the development of suitable chromogenic peptide reagents. Such methods provide a continuous precise assay for endopeptidases. Spectrophotometric methods, however, require relatively pure preparations of enzyme and are not normally suitable for evaluation of endopeptidase activities in crude or particulate samples.

Despite these efforts, at present, the only convenient assay for the biological activity of the botulinum neurotoxins is the mouse lethality test. This test suffers from a number of drawbacks: (1) it is expensive and uses large numbers of laboratory animals, (2) it is non-specific unless performed in parallel with toxin neutralization tests using specific anti-sera, and (3) it lacks accuracy unless large animal groups are used.

There is a clear need for high precision and low complexity assays for determining the potency of botulinum toxins. The present disclosure provides answers this need.

### SUMMARY OF THE DISCLOSURE

The present disclosure is generally drawn to cell-based methods of determining the potency of botulinum neurotoxins (BoNTs) without having to rely on LD₅₀ experimentation on mice.

In some aspects, the disclosure is broadly drawn to a method of determining potency of botulinum neurotoxins (BoNTs), the method comprising: (a) distributing at least two different BoNT samples to at least two containers comprising cells expressing a SNAP25 protein, wherein the first BoNT sample is a reference sample of a known potency and the second BoNT sample is a test sample of unknown potency, (b) incubating the cells with the BoNT for a period of time, (c) determining the ratio of cleaved SNAP25 protein to uncleaved SNAP25 protein corresponding to the reference sample and the test sample, and (d) identifying the potency of the test sample relative to the reference sample.

In some aspects, a third BoNT sample, a quality control sample, of known potency, is distributed to a third container and utilized as a positive control. In some aspects, the relative potency of the test sample is at least 95% as accurate as compared to a murine LD₅₀ assay. In some aspects, (c) comprises subjecting the cleaved and uncleaved SNAP25 proteins to Western blot and densitometric quantification.

In some aspects, the period of time is for at least 6, 12, 16, 20, 24, 32, 40, 48, or 56 hours. In some aspects, the at least two containers each comprise a plurality of wells. In some aspects, the at least two different BoNT samples are serially diluted across the plurality of wells. In some aspects, the at least two containers are tissue culture plates. In some aspects, the at least two containers are 48-, 96-, 384-, or 1536-well plates.

In some aspects, the cells are adhered or attached to the at least two containers. In some aspects, the cells natively express SNAP25. In some aspects, the cells express a heterologous SNAP25. In some aspects, the cells are non-neuronal cells. In some aspects, the cells are genetically modified. In some aspects, the cells are neuronal cells. In some aspects, the neuronal cells are motor neurons.

In some aspects, the cells are treated with a non-proliferation agent. In some aspects, the non-proliferation agent inhibits γ-secretase. In some aspects, the non-proliferation agent is DAPT. In some aspects, a protease inhibitor is added to the at least two containers upon conclusion of (b).

In some aspects, the cells are lysed after incubating the cells with the BoNT. In some aspects, the cells are lysed by sonication. In some aspects, the cells are lysed by addition of a lysis agent. In some aspects, the lysis agent comprises a detergent. In some aspects, the BoNT samples are selected from BoNT/A, BoNT/E, and BoNT/C.

The following detailed description is exemplary and explanatory, and is intended to provide further explanation of the invention.

### BRIEF DESCRIPTION OF THE FIGURES

FIG. 1 depicts an exemplary workflow of the cell based botulinum toxin potency method.

FIG. 2 depicts the workflow for seeding and maintaining of the motor neurons.

FIG. 3 depicts the placing of the motor neurons in a 96-well plate marked with grey. Rows A and H, as well as column 1 and 12 are left empty to avoid an “edge effect.”

FIG. 4 depicts the complete plate setup of an assay with three cell plates. Each toxin unit is added into alternating rows to avoid plate bias. Ref=reference, QC=quality control, and Unk=unknown.

FIG. 5 depicts the schematic overview of the workflow for the Western blotting.

FIG. 6 depicts an image obtained through Western blotting. All 15 lanes from the TGX protein gel are visible. In each lane, two bands of proteins are detected: uncleaved (upper band) and cleaved (lower band) SNAP25. Each lane of protein gel depicted in FIG. 6 is described in Table A.

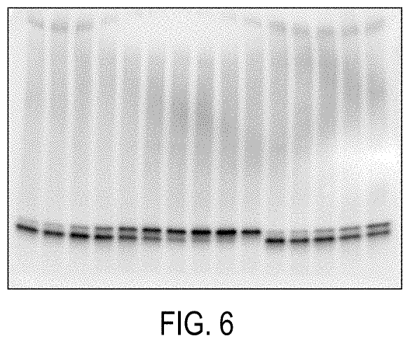

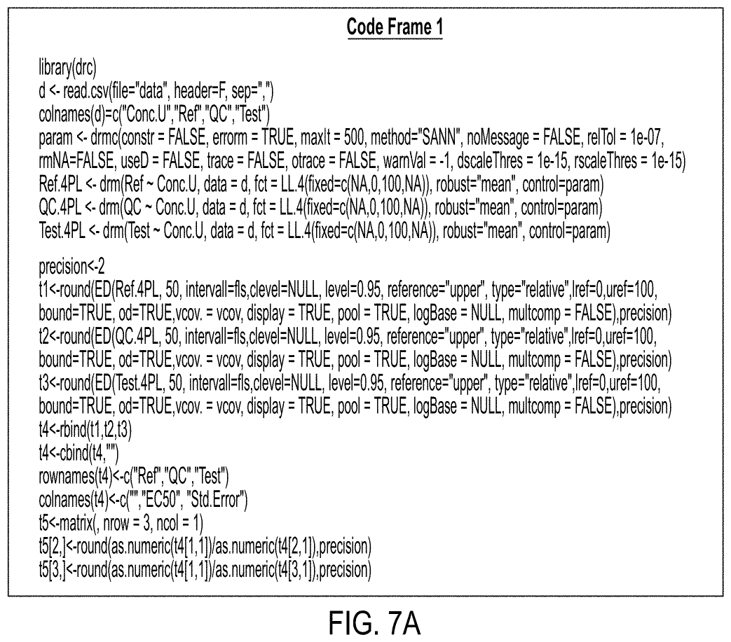

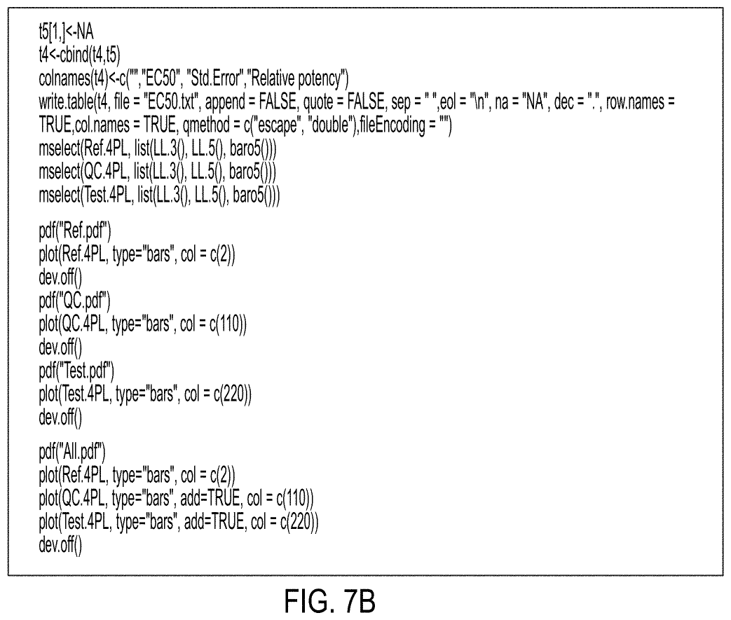

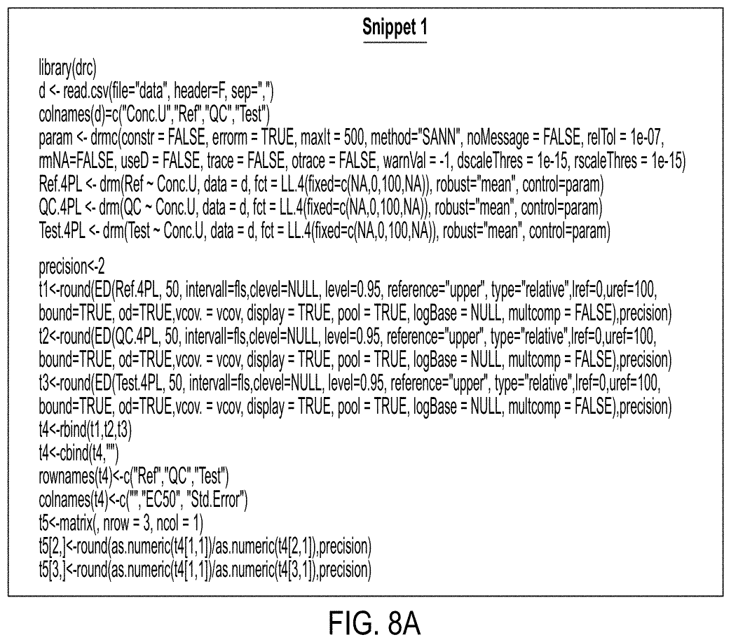

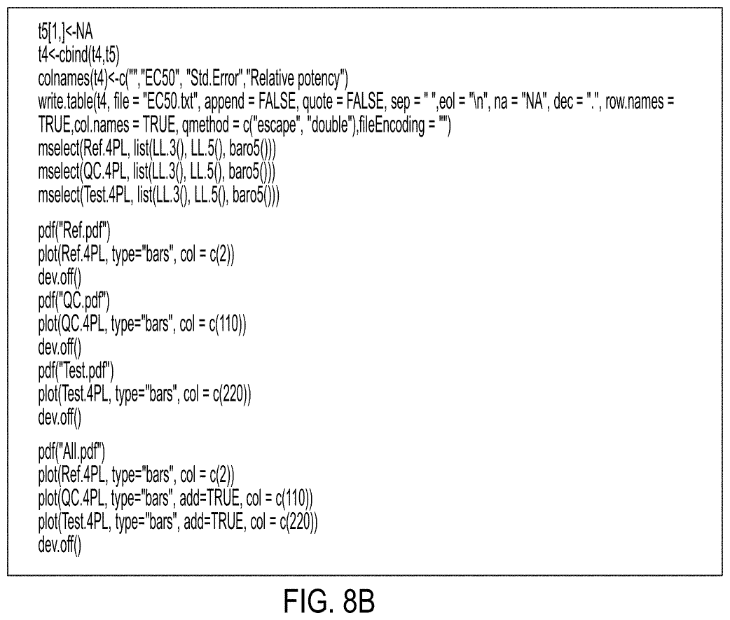

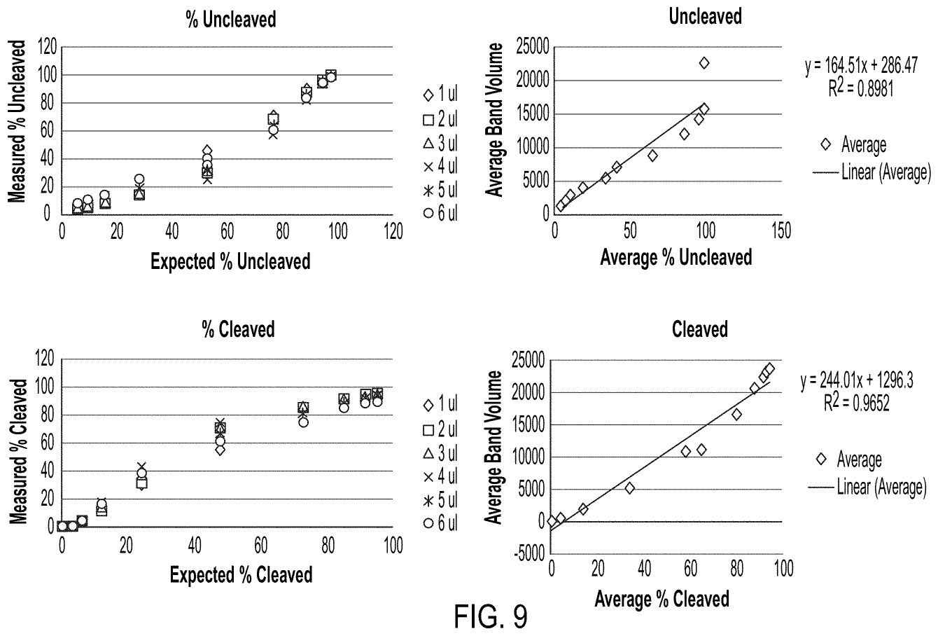

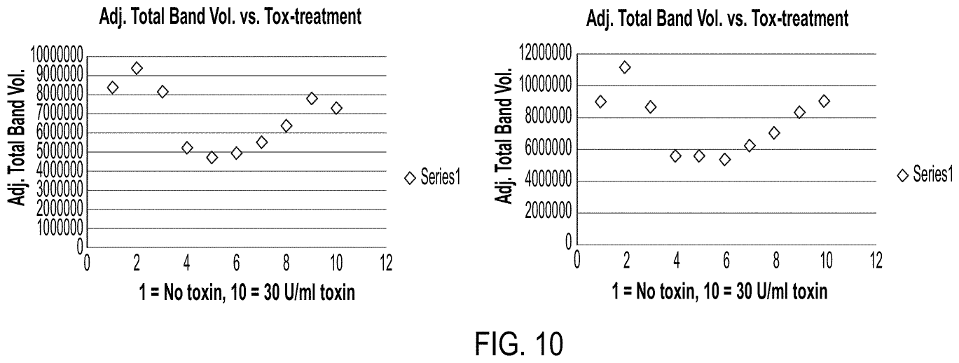

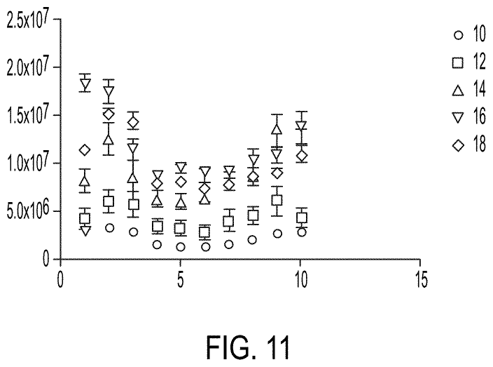

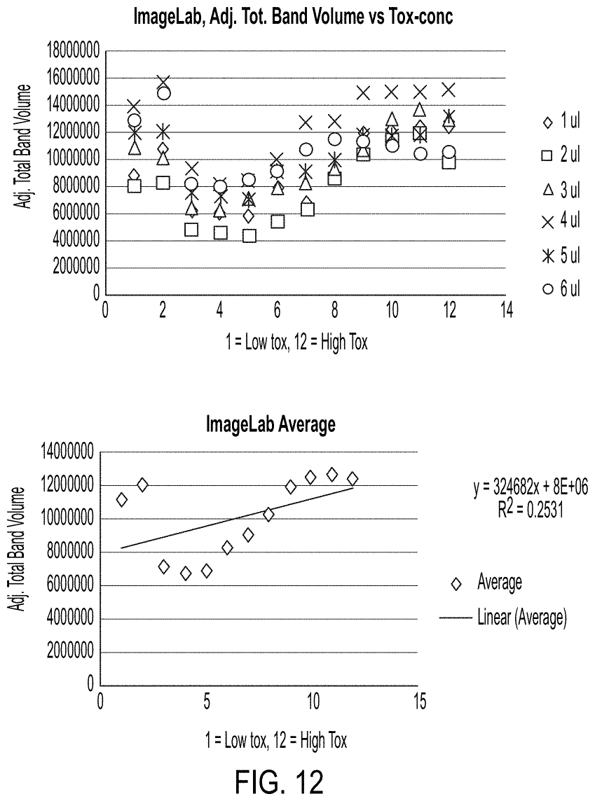

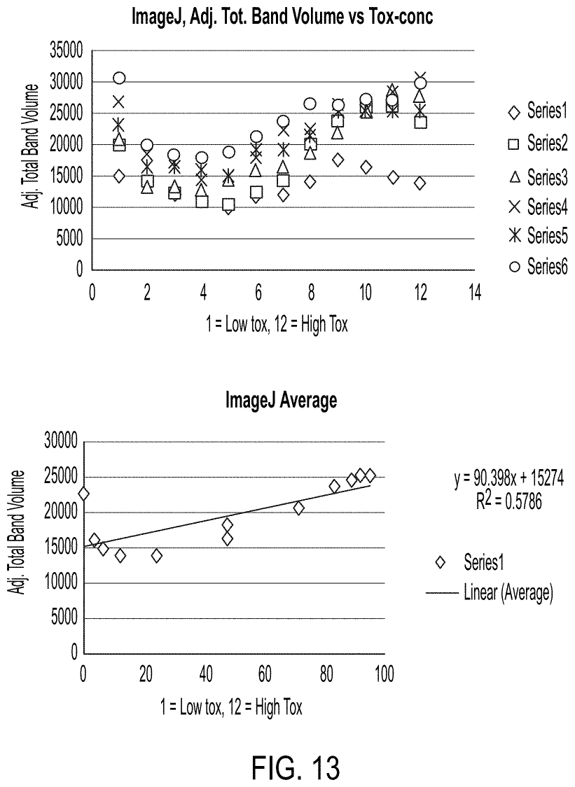

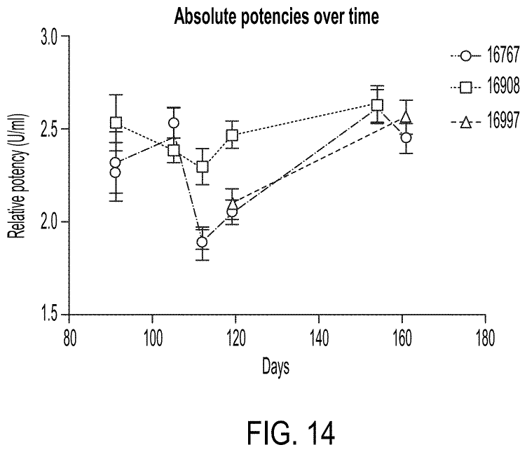

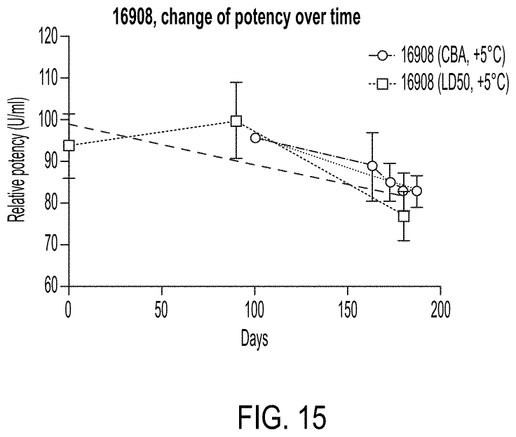

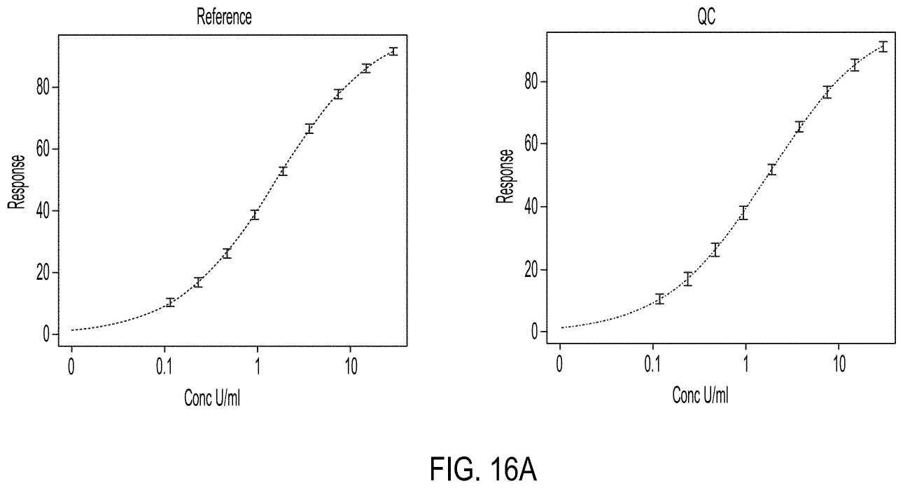

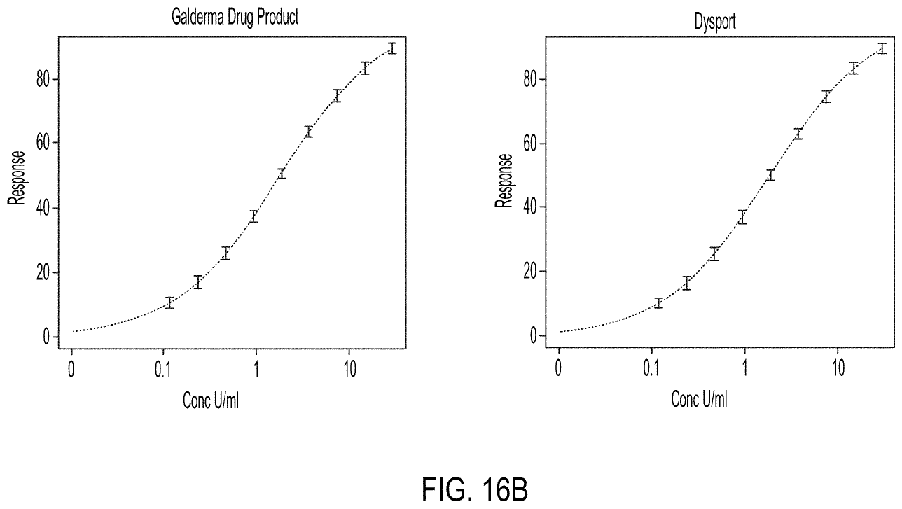

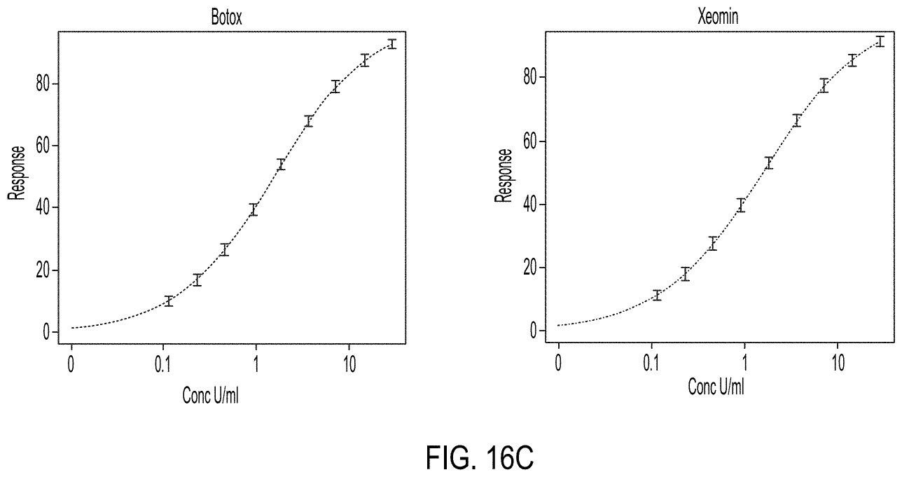

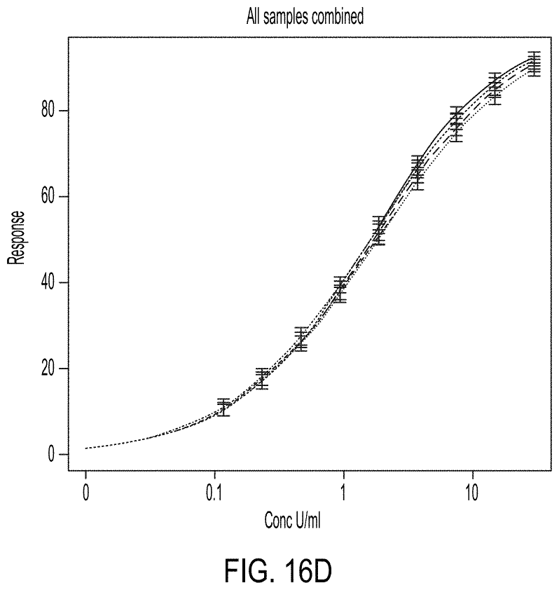

TABLE A
Description of the 15 lanes, from left to right,
of the protein gel depicted in FIG. 6.

| Lane | Description of Contents |
|---|---|
| 1 | SNAP-25 from motor neurons treated with reference Galderma |
| | drug product sample at 0.058 U/ml |
| 2 | SNAP-25 from motor neurons treated with reference Galderma |
| | drug product sample at 0.12 U/ml |
| 3 | SNAP-25 from motor neurons treated with reference Galderma |
| | drug product sample at 0.23 U/ml |
| 4 | SNAP-25 from motor neurons treated with reference Galderma |
| | drug product sample at 0.47 U/ml |
| 5 | SNAP-25 from motor neurons treated with reference Galderma |
| | drug product sample at 0.94 U/ml |
| 6 | SNAP-25 from motor neurons treated with reference Galderma |
| | drug product sample at 1.87 U/ml |
| 7 | SNAP-25 from motor neurons treated with reference Galderma |
| | drug product sample at 3.75 U/ml |
| 8 | SNAP-25 from motor neurons treated with reference Galderma |
| | drug product sample at 7.50 U/ml |
| 9 | SNAP-25 from motor neurons treated with reference Galderma |
| | drug product sample at 15.0 U/ml |
| 10 | SNAP-25 from motor neurons treated with reference Galderma |
| | drug product sample at 30.0 U/ml |
| 11 | SNAP-25 from motor neurons treated with QC Galderma drug |
| | product sample at 0.058 U/ml |
| 12 | SNAP-25 from motor neurons treated with QC Galderma drug |
| | product sample at 0.12 U/ml |
| 13 | SNAP-25 from motor neurons treated with QC Galderma drug |
| | product sample at 0.23 U/ml |
| 14 | SNAP-25 from motor neurons treated with QC Galderma drug |
| | product sample at 0.47 U/ml |
| 15 | SNAP-25 from motor neurons treated with QC Galderma drug |
| | product sample at 0.94 U/ml |

FIG. 7A-7B depict the 3plate.rs script which may be utilized in DRC analysis with R.

FIG. 8A-8B depict an alternative code script for R statistical analysis.

FIG. 9 depicts plots of the measured % cleaved SNAP25 against expected values of % cleaved. Top left panel: % measured uncleaved SNAP25 vs. expected % uncleaved SNAP25, to the right: average band volume vs. average % uncleaved SNAP25. Bottom left panel: % measured cleaved SNAP25 vs. expected % cleaved SNAP25, to the right: average band volume vs. average % cleaved SNAP25.

FIG. 10 depicts the average of Adj. Total Band Col. of experiments 10-18 with gel outliers in the left panel and without gel outliers in the right panel.

FIG. 11 depicts the Adj. total Band Vol. of experiments 10-18 without gel outliers as plotted in GraphPad Prism.

FIG. 12 depicts the ImageLab analysis of Adj. Total Band Vol. of dilution series with DP-buffer control samples and high toxin-treated samples. The dilution series were analyzed with western blot with dilution of the protein samples ranging from 1-6 ul.

FIG. 13 depicts the ImageJ analysis of Adj. Total Band Vol. of dilution series with DP-buffer control samples and high toxin-treated samples. The dilution series were analyzed with western blot with dilution of the protein samples ranging from 1-6 ul.

FIG. 14 depicts the absolute potencies plotted versus the time (in days) these absolute potencies were obtained. Absolute potencies for all samples from experiments 4, 8, 10, 12, 14, 16, 18, and 20.

FIG. 15 depicts the data presented in Table 16, LD₅₀ data in circular data points and cell based data in square data points. Comparison of relative potencies obtained with the LD₅₀ method and cell based method on the same sample at different time points.

FIG. 16A-16D depict the characterized potency of a Reference sample (FIG. 16A, left panel), QC sample (FIG. 16A, right panel), Galderma drug product (FIG. 16B left panel), DYSPORT (FIG. 16B right panel), BOTOX (FIG. 16C left panel), XEOMIN (FIG. 16C right panel), and a merger of the potency data for each of the aforementioned samples. The potency samples were determined according to the methods described herein.

### DETAILED DESCRIPTION OF THE DISCLOSURE

### I. Definitions

While the following terms are believed to be well understood by one of ordinary skill in the art, the following definitions are set forth to facilitate explanation of the presently disclosed subject matter.

The term “a” or “an” may refer to one or more of that entity, i.e. can refer to plural referents. As such, the terms “a” or “an”, “one or more” and “at least one” are used interchangeably herein. In addition, reference to “an element” by the indefinite article “a” or “an” does not exclude the possibility that more than one of the elements is present, unless the context clearly requires that there is one and only one of the elements.

Reference throughout this specification to “one embodiment”, “an embodiment”, “one aspect”, or “an aspect” means that a particular feature, structure or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics can be combined in any suitable manner in one or more embodiments.

As used herein, the terms “about” or “approximately” when preceding a numerical value indicates the value plus or minus a range of 10% of the value.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

As used herein, a “control” is an alternative sample used in an experiment for comparison purpose. A control can be “positive” or “negative.” A “control sample” or “reference sample” as used herein, refers to a sample or reference that acts as a control for comparison to an experimental sample. For example, an experimental sample comprises compound A, B, and C in a vial, and the control may be the same type of sample treated identically to the experimental sample, but lacking one or more of compounds A, B, or C.

As used herein, “SNAP25” and “SNAP-25” refer to a peptide sequence, or fragment thereof, of a human SNAP25 protein (Entrez 6616; UniProt P60880).

As used herein, “VAMP-1” and “VAMP1” refer to a peptide sequence, or fragment thereof, of a human VAMP1 protein (Entrez 6843; UniProt P23763).

As used herein, “VAMP-2” and “VAMP2” refer to a peptide sequence, or fragment thereof, of a human VAMP2 protein (Entrez 6844; UniProt P63027).

As used herein, “VAMP-3” and “VAMP3” refer to a peptide sequence, or fragment thereof, of a human VAMP3 protein (Entrez 9341; UniProt Q15836).

As used herein, “syntaxin” and “syntaxin 1a” refers to a peptide sequence, or fragment thereof, of a human syntaxin protein (Entrez 6804; UniProt Q16623).

As used herein, “botulinum toxin,” “botulinum neurotoxin,” and “BoNT” are used interchangeably to refer to any of the neurotoxic proteins produced by the bacterium Clostridium botulinum. The neurotoxic proteins include botulinum neurotoxin A, B, C, D, E, F, G, and H.

As used herein, “Galderma drug product” refers to a BoNT/A product developed by Applicant.

The present technology is not to be limited in terms of the particular aspects described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

### II. Botulinum Toxins (BoNTs)

BoNTs have been traditionally classified into seven serotypes distinguishable with animal antisera and designated with the letters, A, B, C, D, E, F, G, and H. Molecular genetic analysis has led to the discovery of genes encoding for many novel BoNTs, include subtypes within each of the serotypes, expanding the known genus of BoNTs drastically over the last decade. While the first discovered BoNTs were known to be produced by Clostridium botulinum, multiple Clostridium species produce BoNTs. In some aspects, BoNTs are produced by C. botulinum, C. baratii, C. butyricum, and C. argentinense. In some aspects, BoNT serotypes are selected from A, B, C, D, E, F, G, H. In some aspects, BoNTs enzymatically cleave SNAP25, VAMP1, VAMP2, VAMP3, syntaxin. In some aspects, SNAP25 is cleaved by BoNT/A, BoNT/E, and BoNT/C. In some aspects, VAMP1 is cleaved by BoNT/B, BoNT/F, BoNT/D, BoNT/G, and BoNT/H. In some aspects, VAMP2 is cleaved by BoNT/B, BoNT/F, BoNT/D, BoNT/G, and BoNT/H. In some aspects, VAMP3 is cleaved by BoNT/B, BoNT/F, BoNT/D, BoNT/G, and BoNT/H. In some aspects, syntaxin is cleared by BoNT/C.

In some aspects, the BoNTs are chimeric. In some aspects, chimeric BoNTs are selected from BoNT/DC, BoNT/CD, and BoNT/FA. In some aspects, BonT/A comprises subtypes selected from A1, A2, A3, A4, A5, A6, A7, and A8. In some aspects, BoNT/B comprises subtypes selected from B1, B2, B3, B4, B5, B6, B7, and B8. In some aspects, BoNT/E comprises subtypes selected from E1, E2, E3, E4, E5, E6, E7, E8, E9, E10, E11, and E12. In some aspects, BoNT/F comprises subtypes selected from F1, F2, F3, F4, F5, F6, F7, and F8. In some aspects, BoNT/G comprises subtype G. In some aspects, BoNT/H comprises subtypes selected from H, F/A, and H/A.

In some aspects, the BoNTs are multivalent, such as bivalent and trivalent. In some aspects, multivalent BoNTs comprise BoNT/Ba, BoNT/Bf, BoNT/Ab, BoNT/Af, BoNT/A(B), and BoNT/A2F4F5.

### III. Determining Relative Potency of Botulinum Toxins

The purpose of the cell based potency method is to determine the relative potency between botulinum toxins, such as BoNT/A run in the assay, replacing the LD₅₀ experiments on mice. In some aspects, the methods described herein determine the potency of BoNT/A, BoNT/E, and/or BoNT/C toxins, all of which are capable of cleaving SNAP25. The methods described herein may be applied to any of the BoNTs described herein and their corresponding substrate cleavage partner.

LD₅₀ assays are highly process- and product-specific assays, which is exemplified through the lot-to-lot variability that can be seen in botulinum toxins, and the need for internal standardization for all manufacturers. This variability is further exemplified by the inability to standardize products and formulations from different manufacturers. LD₅₀ assays must be performed for every botulinum toxin composition. The nature of this assay also creates discrepancies, even when the same assay is performed in the same manner between batches. This is because the drug is cultured from bacterial fermentations, which inherently have variability between batches. Taken together, a skilled practitioner would be away that one “unit” of on botulinum toxin preparation is not equivalent to another “unit” from a different composition unless proper LD₅₀ assay controls demonstrated potency equivalence. The methods described herein demonstrate the ability to determine relative potency between different samples, batches, products, and even different botulinum neurotoxin serotypes.

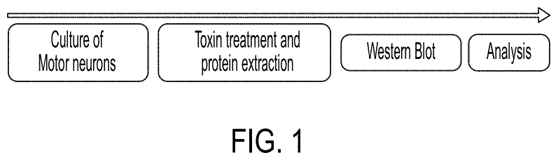

In some aspects, each assay utilizes three different toxin samples, categorized as Reference, Quality Control (QC), and Test. The Test sample has an unknown potency, the Reference is a sample with already established potency. A relative potency is calculated between the QC and Reference samples, and because this is a known value, it can be used as an Assay Acceptance Criteria. The assay is divided into four separate parts as illustrated in FIG. 1. In some aspects, the QC sample is excluded and each assay utilizes two different toxin samples, the Reference sample and Test sample.

In some aspects, the reference sample comprises 10 U/ml, 20 U/ml, 30 U/ml, 40 U/ml, 50 U/ml, 60 U/ml, 70 U/ml, 80 U/ml, 90 U/ml, 100 U/ml, 110 U/ml, 150 U/ml, 200 U/ml, or 300 U/ml of the BoNT. In some aspects, the reference sample comprises about 10 U/ml, about 20 U/ml, about 30 U/ml, about 40 U/ml, about 50 U/ml, about 60 U/ml, about 70 U/ml, about 80 U/ml, about 90 U/ml, about 100 U/ml, about 110 U/ml, about 150 U/ml, about 200 U/ml, or about 300 U/ml of the BoNT. In some aspects, the reference sample comprises between 20 U/ml and 300 U/ml, 20 U/ml and 200 U/ml, 50 U/ml and 150 U/ml, 80 U/ml and 120 U/ml, 90 U/ml and 100 U/ml.

In some aspects, the QC sample comprises 10 U/ml, 20 U/ml, 30 U/ml, 40 U/ml, 50 U/ml, 60 U/ml, 70 U/ml, 80 U/ml, 90 U/ml, 100 U/ml, 110 U/ml, 150 U/ml, 200 U/ml, or 300 U/ml of the BoNT. In some aspects, the QC sample comprises about 10 U/ml, about 20 U/ml, about 30 U/ml, about 40 U/ml, about 50 U/ml, about 60 U/ml, about 70 U/ml, about 80 U/ml, about 90 U/ml, about 100 U/ml, about 110 U/ml, about 150 U/ml, about 200 U/ml, or about 300 U/ml of the BoNT. In some aspects, the QC sample comprises between 20 U/ml and 300 U/ml, 20 U/ml and 200 U/ml, 50 U/ml and 150 U/ml, 80 U/ml and 120 U/ml, 90 U/ml and 100 U/ml.

In some aspects, the test sample is found to have a potency of between 80-130% of the reference sample. In some aspects, the test sample is found to have a potency of between 80-130%, 80-120%, 80-110%, 80-100%, 80-90%, 90-130%, 90-120%, 90-110%, 90-100%, 100-130%, 100-110%, 110-130%, 110-120%, or 120-130% of the reference sample. In some aspects, the test sample is found to have a potency of within 1%, 2%, 3%, 4%, 5%, 6%, 7%, 8%, 9%, 10%, 15%, 20%, 25%, 30%, 35%, 40%, 45%, or 50% of the reference sample. In some aspects, the test sample is found to have a potency of within about 1%, about 2%, about 3%, about 4%, about 5%, about 6%, about 7%, about 8%, about 9%, about 10%, about 15%, about 20%, about 25%, about 30%, about 35%, about 40%, about 45%, or about 50% of the reference sample.

In some aspects, the potency can be determined between 8-200 U/mL. In some aspects, the potency can be determined between 30-200 U/mL. In some aspects, the potency of a sample with greater than 200 U/mL can be determined by diluting the test sample by a factor or 1, 2, 5, 10, or 100, followed by determining the potency of each of each dilution and calculating the potency in view of the dilution factor utilized in diluting the sample. In some aspects, the potency of a sample with greater than 200 U/mL can be determined by serially diluting the test sample by a factor or 1, 2, 5, or 10 in 1, 2, 3, 4, 5, 6, 7, 8, 9, or 10 serial dilutions, followed by determining the potency of each of each dilution and calculating the potency in view of the dilution factor utilized in diluting the sample.

In some aspects, the potency of one or more BoNT products or samples are determined relative to a control sample for which the potency of each of the BoNT products are determined relative to the control sample. In some aspects, the potency of at least 2, 3, 4, 5, 6, 7, 8, 9, or 10 different BoNTs or BoNT samples are determined in parallel. In some aspects, the at least 2, 3, 4, 5, 6, 7, 8, 9, or 10 different BoNTs or BoNT samples are determined in parallel relative to a control sample, yielding a relative potency.

A. Cell culture

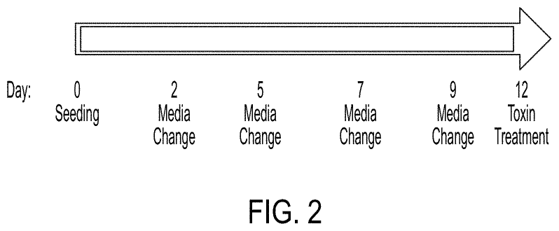

For this potency method, the iCells Motor Neurons (iPSC) from Cellular Dynamic International are used. The workflow for seeding and maintaining of the cells is illustrated in FIG. 2. The cells are generally stored at approximately −150° C., in thaw and use format.

The first step is to thaw and seed the cells into 96-well plates. The cells are thawed using a water bath set to 37° C. After thawing, the cells are diluted in cell media supplemented with the nutrients necessary for the cells' survival. The cells are to be diluted quickly, because of their sensitivity to the DMSO that is present in the media the cells are frozen in, but not too fast, since the cells can be damaged by osmosis. Before adding the cells to the cell plates, the plates are coated with Poly-D-Lysine (PDL) and topped with Geltrex Matrix. This promotes adherence of the cells. The cells are then seeded to the plate by using a multichannel pipet.

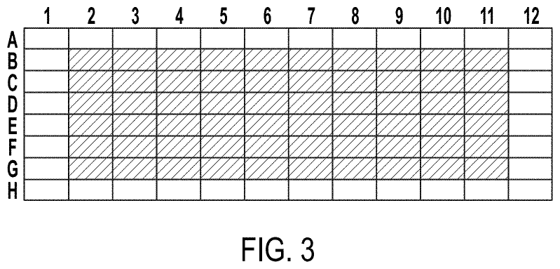

From early experiments it was shown that there is an “edge effect” that affects the well-being of the cells, resulting in less healthy cells in the outermost wells of the plate. Therefore, the cells are seeded in the wells as shown in FIG. 3, in a so-called “inner 60 well” format. One assay consists of three cell plates, as described in FIG. 3.

In order for the motor neurons to thrive, a media exchange is performed generally every two to three days after seeding. In this assay 75% of the media is exchanged on day 2, 5 and 7 after seeding. In some aspects, a non-proliferation agent is added to the cells. In some aspects, the non-proliferation agent exhibits γ-secretase. In some aspects, the non-proliferation agent is DAPT. During this first week, the media contains an agent called DAPT, which keeps the cells from proliferating into fibroblasts instead of motor neurons.

On day 9 after seeding, 50% of media is exchanged and the new media is from now on without DAPT. On day 12 after seeding the cells are treated with toxin. The time required for seeding and maintaining of the cells is summarized in Table 1.

TABLE 1
The required work hours for seeding, maintaining,
and toxin treating the motor neurons.

| Task | Hours of work |
|---|---|
| Seeding | 3 |
| Media change x4 | 1 |
| Toxin treatment | 4.5 |

In some aspects, the cells are subjected to a media exchange at least once per day post-seeding. In some aspects, the cells are subjected to a media exchange at least once every two days post-seeding. In some aspects, the cells are subjected to a media exchange at least once every three days post-seeding. In some aspects, the cells are subjected to a media exchange at least once every four days post-seeding. In some aspects, the cells are subjected to a media exchange no more than 2, 3, 4, or 5 times within a two day span post-seeding.

In some aspects, the media exchange is a 10% media exchange. In some aspects, the media exchange is a 20% media exchange. In some aspects, the media exchange is a 25% media exchange. In some aspects, the media exchange is a 30% media exchange. In some aspects, the media exchange is a 40% media exchange. In some aspects, the media exchange is a 50% media exchange. In some aspects, the media exchange is a 60% media exchange. In some aspects, the media exchange is a 70% media exchange. In some aspects, the media exchange is a 75% media exchange. In some aspects, the media exchange is a 80% media exchange. In some aspects, the media exchange is a 90% media exchange. In some aspects, the media exchange is a 100% media exchange.

In some aspects, the plates in which the cells are seeded into are tissue culture plates. In some aspects, the plates in which the cells are seeding into are coated with a substance that promotes cellular adhesion of the cells to the plates. In some aspects, the plates are selected from 4-well, 8-well, 12-well, 16-well, 24-well, 48-well, 96-well, 384-well, or 1536-well plates. In some aspects, the cells adhere to the plates. In some aspects, the cells do not adhere to the plates. In some aspects, the cells are adhered to or attached to the bottom of the wells of the plates. In some aspects, the cells are adhered to or attached to the bottom and the sides of the wells of the plates.

In some aspects, iCells Motor Neurons (iPSC) from Cellular Dynamics International are utilized for the purpose of performing potency testing on them. The motor neurons are a highly pure population of human neurons derived from induced pluripotent stem cells. In some aspects, the cells grow on PDL+Geltrex matrix coated culture vessels, with media supplemented with DAPT replacement every 2-3 days for the first week and then in media without DAPT replaced every 2-3 days the rest of the culture time. The motor neurons are adherent cells and remain viable for up to or more than 14 days, which are generally stored in −150° C. low temperature freezers.

In some aspects, motor neurons are cultured utilizing one or more of the following media and/or supplements: iCell Neural Base Medium, iCell Neural Supplement A, iCell Nervous System Supplement, poly-D-lysine, Geltrex Basement Membrane Matrix, DAPT (>98%), DMSO (Hybrid Mac), sterile water, 70% ethanol, and 0.4% trypan blue solution.

Preparation of PDL and Geltrex Matrix Cell Culture Vessels

In some aspects, motor neurons grow on a coating of Poly-D-Lysine (PDL) with a fresh layer of Geltrex Matrix on top. In some aspects, the plating is prepared the same day as the cells are thawed.

In some aspects, the PDL-Geltrex Matrix plates are prepared as follows:

1. Add PDL solution in each well in sterile cell culture plates according to Table 2. In some aspects, pre-PDK coated cell culture vessels may be used.

2. Incubate the cell culture plates with the lid closed, at room temperature for at least 1 hour.

3. After incubation with PDL, completely aspirate the solution from each well. Rinse each well twice with sterile water, and aspirate completely. PDL is generally toxic and must be rinsed away.

4. Add the Geltrex Matrix to each well according to table above and cover with the lid. Incubate at room temperature in the safety bench for at least an hour. The Geltrex is aspirated immediately before the cells are added. Prevent the Geltrex surface from drying out.

TABLE 2
Volumes for preparation of plates.

| | Volume of | Volume of water | Volume of |
|---|---|---|---|
| | PDL solution | rinse or PBS | Geltrex |
| Culture Vessel | (mL) | (mL) | (mL) |
| 6-well plate | 1 | 2 | 1 |
| 12-well plate | 1 | 2 | 0.8 |
| 24-well plate | 0.5 | 1 | 0.5 |
| 96-well plate | 0.1 | 0.2 | 0.1 |

Prepare Motor Neuron Medium

In some aspects, the cell medium for Motor Neurons is supplied by Cellular Dynamics International together with the cells and consists of iCell Neural Base Medium (1 bottle, ˜100 mL), iCell Neural Supplement A (1 vial with ˜2 mL) and iCell Nervous System Supplement (1 vial with ˜1 mL). When prepared utilizing sterile techniques, the complete medium is then stable for 2 weeks when stored at 4° C.

Motor Neurons grow in complete medium supplemented with DAPT the first 9 days, and on subsequent media changes 50% of the DAPT-containing media is replaced with just complete media for the remaining of the culture time. In some aspects, the media is prepared as follows:

1. Thaw the supplements overnight at 4° C. or at room temperature protected from light for 30 min until no visible ice is in the tube.

2. Add the entire content of the supplements to the iCell Neural Base Medium bottle.

3. Rinse the supplement tubes with ˜1 mL medium each.

4. To make medium that can be used during the first week of culture, move 50 mL of the complete maintenance medium to a sterile 50 mL centrifuge tube. Write the date on the medium bottle and store in the fridge protected from light.

5. Dissolve DAPT in DMSO to achieve a concentration of 20 mM (8.6 mg/mL) by calculating the volume of sterile DMSO that needs to be added to the vial of DAPT. In some aspects, DAPT inhibits differentiation of hIPSC.

6. To the 50 mL tube of medium, add 12.5 μl of DAPT to achieve a final concentration of 5 μM.

7. Sterile filter this through a 0.22 μm sterile filter unit. Store at 4° C. protected from light.

8. The medium is to be equilibrated to room temperature when thawing cells as well as for media changes.

Thawing and Plating Cells

In some aspects, cells are to be plated at approximately 1×10⁵ viable cells/cm². In some aspects, the cells are plated at about 1×10⁴, 2×10⁴, 4×10⁴, 6×10⁴, 8×10⁴, 1×10⁵, 2×10⁵, 4×10⁵, 6×10⁵, or 8×10⁵ viable cells/cm².

Table 3 provides exemplary dell densities and plating volumes for motor neurons.

TABLE 3
Cell densities and plating volumes for motor neurons.

| Culture | Surface area | | Plating Volume | | Cell number | Cell number |
|---|---|---|---|---|---|---|
| Vessel | (cm²) | | (mL) | | (cells cm²) | (cells/mL) |
| 6-well | 9.6 | cm² | 2 | mL | 9.6 × 10⁵ | 4.2 × 10⁴ |
| plate | | | | | | |
| 12-well | 3.8 | cm² | 1 | mL | 3.8 × 10⁵ | 1.25 × 10⁵ |
| plate | | | | | | |
| 24-well | 2 | cm² | 0.5 | mL | 2.5 × 10⁵ | 2.5 × 10⁵ |
| plate | | | | | | |
| 96-well | 0.32 | cm² | 0.2 | mL | 3.2 × 10⁴ | 6.25 × 10⁵ |
| plate | | | | | | |

In some aspects, the cells are counted once thawed and added to the plates for culturing.

Maintaining Cells

In some aspects, the cells, including motor neurons, can be maintained at least for 2 weeks. The first week, use Complete Maintenance Medium+DAPT and change 75% every 2-3 days. After 1 week of culture, perform 50% medium exchange with only Complete Maintenance Medium every 2-3 days.

In some aspects, neuronal cell lines are utilized in the methods described herein. In some aspects, the neuronal cell lines are human neuronal cell lines. In some aspects, the neuronal cell lines are mammalian neuronal cell lines. In some aspects, the neuronal cell lines are non-human cell lines. In some aspects, the neuronal cell lines are selected from the following: motor neuron, astrocyte, astroglia, neuroblast, dopaminergic neuron, cortical neuron, and neuron. In some aspects, the neuronal cell lines are motor neurons, interneurons, or sensory neurons.

In some aspects, the methods described herein utilize cells that express SNAP25. In some aspects, the methods described herein may utilize non-neuronal cell lines that express SNAP25. In some aspects, the cells natively express SNAP25. In some aspects, the cells are modified to express a heterologous SNAP25. In some aspects, the SNAP25 is wild type. In some aspects, the SNAP25 is modified.

### B. Toxin Treatment and Protein Extraction

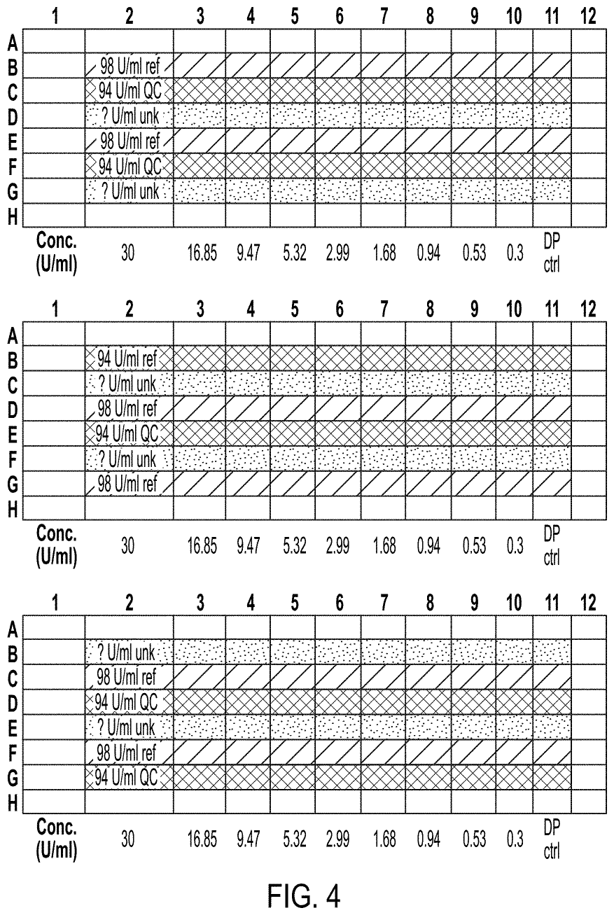

In some aspects, toxin-treatment is performed on one cell plate at a time. In some aspects, the first step when toxin-treating the cells, is to collect 65% of the cell media from the culture wells. New media is added to the collected media and the mix is used for toxin dilution. The media collected from the culture is necessary to keep, since it contains vital substances created by the cells and 100% new media for toxin treatment would damage the cells. The cell media suspension is added to a 96-well mixing plate, mimicking the setup of the 96-well plate with the culture vessels. In the mixing plate the toxin samples are added to the first column of each row, and the toxin is then serial diluted throughout all the media filled columns. The rows which the reference, QC and unknown toxin units are added to are alternated over the three plates in the assay. When the dilution is finished, the remaining cell media in the culture vessels is discarded and replaced by the toxin containing media, column by column. An example of the final plate setup is shown in FIG. 4.

In some aspects, after toxin addition, the cells are incubated for 48 hours, allowing the toxin to exert its effect before the protein is extracted.

In some aspects, the cells are incubated with the toxin for at least 4 hours, at least 12 hours, at least 16 hours, at least 20 hours, at least 24 hours, at least 28 hours, at least 32 hours, at least 36 hours, at least 40 hours, at least 44 hours, at least 48 hours, at least 52 hours, at least 56 hours, at least 60 hours, at least 64 hours, at least 68 hours, or at least 72 hours.

In some aspects, the cells are incubated with the toxin for 4-96 hours, 24-96 hours, 48-96 hours, 4-72 hours, 4-48 hours, 4-24 hours, 24-96 hours, 24-72 hours, 24-48 hours, 48-96 hours, 48-72 hours, 36-56 hours, or 44-52 hours.

In some aspects, the cells are lysed by mechanical perturbation. In some aspects, the cells are lysed with a chemical that disrupts cell walls. In some aspects, the cells are lysed with a detergent. In some aspects, the lysed cells are separated from the resulting supernatant by centrifuge. In some aspects, the lysed cells are separated from the resulting supernatant by filtration.

In some aspects, during protein extraction all cell media is discarded from the cell plate. A cell lysis buffer called RIPA is then added to the wells, causing the cells to lyse, liberating the proteins. The RIPA buffer is supplemented with a protease inhibitor, to keep the proteins intact. After the addition of the cell lysis buffer, the cell plates are agitated to make the cells detach from the bottom of the dish. Finally the lysate is transferred to a PCR-plate for storage in −20° C. In some aspects, the total acquired time for toxin treatment and protein extraction is approximately 4.5 hours, as represented in Table 1.

Preparations

In some aspects, serial dilution of the Galderma drug product, a botulinum neurotoxin type A, is required. In some aspects, the Galderma drug product is a liquid formulation with a nominal concentration of 200 Units/ml (U/ml) or less.

In some aspects, the BoNT has a nominal concentration of 20 U/ml, 25 U/ml, 30 U/ml, 35 U/ml, 40 U/ml, 45 U/ml, 50 U/ml, 55 U/ml, 60 U/ml, 65 U/ml, 70 U/ml, 75 U/ml, 80 U/ml, 85 U/ml, 90 U/ml, 95 U/ml, 100 U/ml, 110 U/ml, 120 U/ml, 140 U/ml, 150 U/ml, 160 U/ml, 170 U/ml, 180 U/ml, 190 U/ml, 200 U/ml, 210 U/ml, 220 U/ml, 230 U/ml, 240 U/ml, or 250 U/ml.

In some aspects, the BoNT has a nominal concentration of about 20 U/ml, about 25 U/ml, about 30 U/ml, about 35 U/ml, about 40 U/ml, about 45 U/ml, about 50 U/ml, about 55 U/ml, about 60 U/ml, about 65 U/ml, about 70 U/ml, about 75 U/ml, about 80 U/ml, about 85 U/ml, about 90 U/ml, about 95 U/ml, about 100 U/ml, about 110 U/ml, about 120 U/ml, about 140 U/ml, about 150 U/ml, about 160 U/ml, about 170 U/ml, about 180 U/ml, about 190 U/ml, about 200 U/ml, about 210 U/ml, about 220 U/ml, about 230 U/ml, about 240 U/ml, or about 250 U/ml.

In some aspects, methods are designed for treatment of a 96-well plate where the edge wells are excluded, yielding a so called inner 60 well design. The dilution scheme is quarter log serial dilution, with two rows for a reference (REF) sample, two rows for a quality control (QC) sample and two rows for a Test sample.

In some aspects, the botulinum toxin is quickly deactivated using 0.2 M NaOH or hypochloride solution, such as ProChlor. These work by degrading the toxin.

Remove toxin vial stored at +4° C. and place it in room temperature 30 min prior to treatment or 1 h prior to treatment if stored at −80° C. Store the vials in dark and handle them with nitrile gloves on. Thaw a 15 mL aliquot of DP-buffer in room temperature 1 h prior to treatment.

Remove cell medium from +4° C. and let it equilibrate to room temperature in dark for 30 min.

Toxin Dilutions

Option 1

Put six tips onto an 8-channel pipette and collect 130 μL cell medium from each well in the cell plate to a reservoir (total of 7.8 mL). Move the medium to a 50 mL falcon tube and add 13 mL fresh medium to it. Mix by turning the tube upside down twice. Put six tips onto the 8-channel pipette and add 300 μL cell medium to nine columns (column 1-9; 6 wells each) in the storage plate. Add additional 179.2 μL medium to column 1.

Open the vial of toxin with the decapper. If toxin contaminates the outer latex/vinyl gloves when removing the lid, change to a new pair. Pour DP-buffer in a reservoir. Before adding the toxin, set the single channel pipette to 200 μL and pre-wet the pipette tip five times with DP-buffer. Be careful to not let any DP-buffer remain in the tip before pipetting toxin. Thereafter, pipette the toxin up and down once and then add 200 μL to the correct well in column 1 on the storage plate. Set another single channel pipette to 5.2 μL and repeat the procedure.

Put six tips on an 8-channel pipette and set it to 300 μL, pre-wet the tips with DP-buffer five times and mix column 1 ten times. Lift 300 μL from column 1 to column 2 and repeat the procedure. Lift from column 2 to column 3, mix, and repeat until all wells are diluted.

Option 2

Put six tips onto an 8-channel pipette and collect 130 μL cell medium from each well in the cell plate to a reservoir (total of 7.8 mL). Move the medium to a 50 mL falcon tube and add 13 mL fresh medium to it. Mix by turning the tube upside down twice. Mix 70% cell medium with 30% DP-buffer by adding 90 μL DP-buffer and 210 μL cell medium in column 2-9. To column 1, add 479.2 μL cell medium.

Open the vial of toxin with the decapper. If toxin contaminates the outer latex/vinyl gloves when removing the lid, change to a new pair. Pour DP-buffer in a reservoir. Before adding the toxin, set the single channel pipette to 200 μL and pre-wet the pipette tip five times with DP-buffer. Be careful to not let any DP-buffer remain in the tip before pipetting toxin. Thereafter, pipette the toxin up and down once and then add 200 μL to the correct well in column 1 on the storage plate. Set another single channel pipette to 5.2 μL and repeat the procedure.

Put six tips on an 8-channel pipette and set it to 300 pre-wet the tips with DP-buffer five times and mix column 1 ten times. Lift 300 μL from column 1 to column 2 and repeat the procedure. Lift from column 2 to column 3, mix, and repeat until all wells are diluted.

In some aspects, the toxins are serially diluted by a factor of 10 for each dilution. In some aspects, the toxins are serially diluted by a factor of 5 for each dilution. In some aspects, the toxins are serially diluted by a factor of 2 for each dilution.

Toxin Treatments and Incubation

In some aspects, remove all cell medium from one column in the cell plate with an 8-channel pipette (5-100 μL) and slowly add 200 μL the toxin dilution with another 8-channel pipette (30-300 μL), with tips pre-wet five times in DP-buffer. The last column (nr 11) is kept as DP-ctrl. Remove 60 μL cell medium and add 60 μL DP-buffer. Incubate the cell plates for 48 h in the incubator (95% 02; 5% CO₂).

Cell Lysis and Protein Extraction from Cell Culture

In some aspects, the steps for protein extraction from cells must be carried out at 2-8° C. Dissolve one tablet protease phosphatase inhibitor per 10 mL RIPA buffer in a falcon tube. Carefully discard the medium in the cell culture plates with an 8-channel pipette. Add 120 μL ice cold RIPA buffer supplemented with inhibitor. Agitate the contents at 200 rpm for 30 min and at 4° C. Pipette the solution up and down ten times (avoid bubbles), collect the protein in fresh PCR-plates and directly store samples in −20° C.

### C. Determining Amount of Cleaved and Uncleaved SNAP25 Proteins

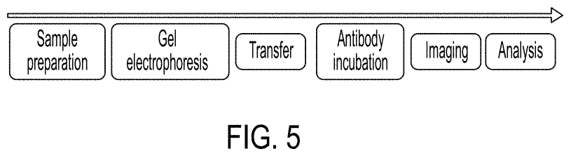

In some aspects the Western blot is used as a tool for visualization of proteins in order to quantify the ratio between the cleaved and uncleaved SNAP25 proteins in the protein samples from the cell cultures. The ratio between the proteins is used to calculate the EC50 values and thereby the potencies of the reference, QC and unknown toxin units, as described in the section Data analysis. The Western blot technique can roughly be divided into six parts, namely; sample preparation, gel electrophoresis, transfer, antibody incubation, imaging and analysis. A schematic imaged of the work flow is illustrated in FIG. 5. The sample preparation is to prepare the proteins for separation by gel electrophoresis. After the separation the proteins are transferred, or blotted, from the gel to a membrane. The membrane is then incubated with antibody to enable the detection of the proteins of interest, after which the membranes are imaged and then analyzed through densitometric quantification.

The frozen protein samples are thawed and then prepared for separation by denaturation through addition of sample buffer and heat appliance. The sample buffer includes sodium dodecyl sulfate (SDS), which denaturizes the proteins and give them a net negative charge and 2-mercaptoethanol that reduces the intra- and intermolecular bonds and breaks disulfide bonds, making the proteins lose their tertiary structure. By denaturizing the proteins, it is ensured that they have a similar charge to mass ratio and structure, so that they will be separated exclusively by their size in the gel electrophoresis.

The denaturized samples are loaded into wells on the top of TGX protein gels. Every gel contains 15 separate wells, all connected to a lane leading through the gel. The protein samples are loaded row-wise from high to low toxin treatment, onto the gels. Each gel holds 15 samples, and so all ten samples from the first row in the protein plate are loaded on the first gel, together with the five first samples from the second row. Then the last five samples from the second row are loaded onto the second gel, together with all ten samples from the third row, and so on. Hence, twelve gels are needed for one complete assay.

After separation of the proteins by gel electrophoresis, the proteins are transferred to membranes, i.e. blotted. In order to visualize the proteins two antibodies are used; a primary antibody that only recognizes whole and cleaved SNAP25 (S-9684 from Sigma Aldrich), and a secondary antibody that recognizes the primary antibody, enabling detection. Before applying the primary antibody, the membrane needs to be blocked using blocking buffer to reduce the unspecific binding of the antibody. After blocking, the primary antibody, dissolved in blocking buffer, is applied and the membrane is incubated overnight to allow the antibody to attach to the proteins. The membrane is then washed using a pH-stable buffer, to remove all excessive antibodies, before application of the secondary antibody. After incubation with the secondary antibody, the membrane is washed again before development.

When developing the images of the proteins, the membranes are incubated with a luminol/peroxidase substrate. The secondary antibody is conjugated with a horse radish peroxidase enzyme that catalyzes an oxidation reaction between luminol and peroxidase, resulting in luminol emitting light, a chemiluminescence. By using a CCD camera the emitted light is detected, with the intensity corresponding to the amount of protein present. A representative image obtained from a western blot membrane is shown in FIG. 6.

In some aspects, the methods described herein require the separation of cleaved SNAP25 from uncleaved SNAP25 for comparison purposes. In some aspects, the identification of cleaved from uncleaved SNAP25 is performed via capillary (gel-free) Western blotting, native gel electrophoresis followed by immunoblotting, denatured gel electrophoresis followed by standard Westering blotting, 2D gel electrophoresis followed by immunoblotting, and microscale Western blotting.

In some aspects, alternatives to Western blots may be utilized to determine the ratio of cleaved SNAP25 to uncleaved SNAP25, such as microscale Western blot (U.S. Pat. No. 9,182,371) or gel-free Western blot (U.S. Pat. No. 9,523,684). In some aspects, quantifying the cleaved and uncleaved SNAP25 is performed via antibody capture of the cleaved and uncleaved SNAP25, purifying the antibody captured proteins via affinity purification and/or HPLC and further determining the amount of cleaved and uncleaved SNAP25.

Detecting SNAP25 Protein with Western Blot (WB)

In some aspects, WB is utilized as a detection method for a cell based assay (CBA) to detect the SNAP25 protein (both cleaved and non-cleaved forms) after treatment with botulinum neurotoxin A in cell cultures.

In some aspects, the WB technique can roughly be divided into six parts; sample preparation, gel electrophoresis, transfer, antibody incubation, imaging and analysis. The sample preparation is to prepare the proteins for separation by gel electrophoresis. After the separation the proteins are transferred, or blotted, from the gel to a membrane. The membrane is then incubated with antibody to enable the detection of the proteins of interest, after which the membranes are imaged and then analyzed through densitometric quantification. After imaging the membranes are discarded.

In some aspects, each potency determination generates three cell plates with 60 protein samples apiece that are to be detected using WB. The gels used for the separation holds 15 protein samples, and thus 12 gels are needed for one complete assay (four for each cell plate). In order to avoid thawing of the samples several times, all samples on one cell plate should preferably be run at the same time. The following method generally describes the procedure for running one gel. However, all amounts of solutions can be multiplied if more cell plate samples are run in one day.

Electrophoresis (Day 1)

Sample preparation and gel electrophoresis. In some aspects, the steps involve samples containing 2-Mercaptethanol, including gel electrophoresis, are performed in a ventilated hood. The wells in the gel can contain 15 μl/well, however, a total volume of 10 μl/well is used.

1. Switch on the heat-block to 95° C. and thaw the frozen protein samples on ice for about 45 min.

2. While the protein samples are thawing, prepare the sample buffer and the running buffer:

2a. Start by labeling the Eppendorf tubes, one for each sample that will be run, including one for the sample buffer. 15 samples are run on each gel and four gels at a time.

2b. Prepare sample buffer in a ventilated hood by adding 2-Mercaptoethanol to 2× Laemmli Sample Buffer in volumes indicated in Table 4 below into the designated Eppendorf tube, and make sure to mix well by pipetting up and down 5 times using a 100 μl pipet. Add 4 μl sample buffer to each Eppendorf tube labelled for protein samples. All tips and tubes that have been in contact with 2-Mercaptoethanol are put in a separate waste for 2-Mercaptoethanol.

2c. Prepare the Running Buffer according to Table 5. Approximately 750 ml Running Buffer in each tetra cell is used. The running buffer is kept in room temperature.

TABLE 4
Volumes used for sample buffer preparation.

| | Sample Buffer preparation | | |
|---|---|---|---|
| | Total volume | 2x Laemmli | 2-Mercaptoethanol |
| Number of gels | (μl) | (μl) | (μl) |
| 2 | 150 | 142.5 | 7.5 |
| 4 | 300 | 285 | 15 |
| 6 | 400 | 380 | 20 |
| 8 | 500 | 475 | 25 |
| 12 | 780 | 741 | 39 |

TABLE 5
Running buffer preparation.

| | Running Buffer preparation | | |
|---|---|---|---|
| | Total Volume | Deionized water | 10x Running buffer |
| Number of gels | (mL) | (mL) | (mL) |
| 2 | 1000 | 900 | 100 |
| 4 | 2000 | 1800 | 200 |
| 6 | 3000 | 2700 | 300 |
| 8 | 4000 | 3600 | 400 |
| 12 | 5000 | 4500 | 500 |

3. When the protein samples are thawed, pipette the protein samples up and down five times, using a 100 μl 12 channeled multi pipet for one row at the time, to mix the protein sample before transferring from cell plate. Work in hood! Add 6 μl of protein sample in the Eppendorf tubes.

4. Incubate the samples for 5 min at 95° C. in a heat-block, to denature the proteins.

5. Meanwhile, prepare the gels;

5a. Open the gel packages and remove the green tape on the bottom of each gel before inserting them into the chamber. If only one gel is used, use the artificial plastic gel to close the chamber. Throw the packages for the gels in a waste bin.

5b. Fill the inner gel chamber to the edge with running buffer. Make sure that the gel chamber is sealed tight and that no buffer is leaking. Then, add running buffer to the marking for 2 gels on the Tetra Cell. The gels can also be prepared this far while the protein samples are thawed.

5c. Remove the green plastic comb on top of the gels and use a Pasteur pipet to wash the wells two times.

6. Take the samples from the heating block. Pipette the samples up and down twice from the bottom of the Eppendorf tube). The 10 μl of the protein samples are loaded row-wise from high to low toxin treatment. Each gel holds 15 samples, and so all ten samples from the first row in the plate are loaded on the first gel, together with the five first samples from the second row. Then the last five samples from the second row are loaded onto the second gel, together with all ten samples from the third row, and so on. No ladder is used.

7. Place the top in the gel chamber and run the electrophoresis 150 V for 90 min. Make sure that the electrophoresis starts by checking for bubbles emerging from the bottom of the gel cassette.

8. Prepare the Blocking Buffer according to Table 6. The solution is kept in room temperature until the first blocking step, then at 4° C.

TABLE 6
5% blocking buffer preparation.

| | Amount of 5% Blocking Buffer | |
|---|---|---|
| Number of gels | (Blocking Buffer, 1XTTBS) | |
| 2 | 100 ml | (5 g, 100 ml) |
| 4 | 150 ml | (7.5 g, 150 ml) |
| 6 | 250 ml | (12.5 g, 250 ml) |
| 8 | 300 ml | (15 g, 300 ml) |
| 12 | 500 ml | (25 g, 500 ml) |

Transfer (Day 1)

1. Prepare for transfer before the electrophoresis is done by taking out the Trans-Blot Turbo Transfer Packs needed from the fridge, one pack is needed per gel. Also get the spatula, rolling pin and the scalpel blade. Take out a cassette from the Trans Blot Turbo Transfer System and remove the top. When the electrophoresis is finished, take out the tetra cell from the hood, detach the gel cassettes and put them pack in the hood.

2. Put the Trans Blot Turbo Transfer pack and cassette in the ventilated hood. Open the transfer pack from the upper right corner and take out the “bottom”-stack, using your hand, and put it in the transfer cassette. Do not invert the stack when moving it to the cassette, lift it as it is in the transfer pack. The membrane is on top of the bottom stack, so try to touch it as little as possible. Use the spatula to break the plastic cover around the gel, where to put the spatula is marked with arrows. Cut off additional gel residues at the top and bottom of the gel using the scalpel blade. Align the bottom of the wells at the top and the dark front line at the bottom of the gel cassette. Try to work fast, if the gel dries out it will break more easily. Wet gloves with running buffer to keep the gel from sticking to the gloves. Lift the gel to the bottom stack in the cassette using your hands. Then lift the “top”-stack from the transfer pack on top of the gel. Use a rolling pin to remove any air-bubbles from the stack-gel composition. Close the cassette with the top cassette. Make sure the top cassette is in “locked” position. The package for the transfer pack is discarded in a waste bin.

3. Use the program TGX Turbo Transfer in the Turbo Transfer System. Insert the cassette in the upper (A) or lower (B) bay. Switch on the Turbo Transfer System. On the home screen, chose the “Turbo” protocol, then chose “1 MINI TGX”. The protocol will be set for a 3 min run with 2.5 A and 25V. Chose A- or B-run, depending on in which bay the cassette is in, to start the run. When the run is complete, the proteins have been transferred to the PVDF membrane.

4. After transfer, the membrane is pre-blocked in 15 ml of blocking buffer for 1 h at agitation (30 rpm, 05 degrees angle) in RT in a small plastic chamber.

4a. Start by pouring the 15 ml of blocking buffer in the plastic chamber.

4b. Take off the top cassette and discard the top stack and the gel in a waste bin.

4c. Carefully lift the membrane with one hand and cut the top of the membrane to fit it into the chamber, the membrane should be free-floating. Also cut off the upper corner of the side of the membrane where the first well of the gel was blotted, to keep track of what is right and left and up and down of the membrane. However, touch the membrane as little as possible. If the transfer was successful, faint transparent traces of the wells/lanes in the gel is visible.

4d. Put the membrane in the blocking buffer when finished and put the plastic chamber on the shaker.

5. The bottom stack is then discarded in the waste bin. Wipe off remaining transfer buffer from the cassette using paper towels and clean the cassette with deionized water. Wipe off the most of the water with paper towels and leave the cassette disassembled for drying for a couple of hours before it is inserted back into the Turbo Blot Transfer machine.

Primary Antibody Incubation

After blocking, incubate the membrane with primary antibody for 10 minutes before the blocking is done, take out the blocking buffer from the fridge and the primary antibodies from the freezer. When the antibodies are thawed, after a few minutes in RT, dilute the primary antibodies in blocking buffer. Anti-SNAP25 (S9684) is diluted 1:1000 and anti-B-actin (A1978) 1:2000, in volumes given in Table 7. Gently vortex the diluted antibody for about two seconds. The anti-B-actin antibody may be skipped. It is not needed for the analysis, but can be included for troubleshooting the assay.

Cut one reaction folder in four, a quarter folder is enough for one membrane. Seal one side of the cut out folder piece using the bag sealer.

When the blocking of the membrane is done, carefully lift the membrane into the prepared folder using tweezers. Put the membrane as close as possible to the sealed side of the folder. Avoid dragging the membrane along the plastic.

Seal two more sides of the plastic folder using the bag sealer. Seal as close to the membrane sides as possible, without sealing on the membrane. Pour the diluted antibodies into the folder.

Carefully remove all large air-bubbles in the antibody solution using fingers. Avoid squeezing the membrane too hard. When no large bubbles are left (small air-bubbles that can easily move around are alright), seal the last side of the plastic folder with the bag sealer. Put the enclosed membrane on a shaker set to 100 rpm with infinity setting (no timer) at 4° C. Leave the membranes for overnight incubation.

TABLE 7
Primary antibody solution preparation.

| | Amount of Primary antibody solution | |
|---|---|---|
| Number of gels | (anti-SNAP/anti-B-actin, Blocking Buffer) | |
| 2 | 10 ml | (10 μl/5 μl, 10 ml) |
| 4 | 20 ml | (20 μl/10 μl, 20 ml) |
| 6 | 30 ml | (30 μl/15 μl, 30 ml) |
| 8 | 40 ml | (40 μl/20 μl, 40 ml) |
| 12 | 60 ml | (60 μl/30 μl, 60 ml) |

Secondary Antibody Incubation

Prepare a small plastic chamber for washing of the membrane by filling it with 20 ml of 1×TTBS.

Cut open the plastic bag with the membrane using scissors. Carefully lift the membrane with tweezers to the prepared plastic chamber. The membrane should be free-floating, otherwise add more 1×TTBS. Wash the membrane for 10 min in RT on agitation (30 rpm, 05 degrees angle).

After 10 minutes, gently pour off the 1×TTBS into a plastic chamber used for waste. Quickly refill the plastic chamber with 20 ml of 1×TTBS, repeat after 10 more minutes (in total 3×10 minutes of washing).

When the last wash has been started, prepare the secondary antibodies, according to Table 8. Use Anti-Rabbit-HRP for the SNAP25 antibody (S9684) and Anti-Mouse-HRP for B-actin (A1978), both diluted 1:10 000 in 5% Blocking Buffer. For one membrane, add 1.5 μl of both antibodies to 15 ml of blocking buffer.

TABLE 8
Secondary antibody solution preparation.

| | Amount of Secondary antibody solution | |
|---|---|---|
| Number of gels | (anti-Rabbit/anti-Mouse, Blocking Buffer) | |
| 2 | 30 ml | (3 μl/3 μl, 30 ml) |
| 4 | 60 ml | (6 μl/6 μl, 60 ml) |
| 6 | 90 ml | (9 μl/9 μl, 90 ml) |
| 8 | 120 ml | (12 μl/12 μl, 120 ml) |
| 12 | 180 ml | (18 μl/18 μl, 180 ml) |

Once the last wash is finished, pour off the 1×TTBS into the waste chamber and add the secondary antibody dilution to the plastic chamber with the membrane. Incubate the membrane in secondary antibody at RT on agitation (30 rpm, 05 degrees angle) for 1 h.

When the incubation with secondary antibody is finished, pour off the antibody solution into the waste chamber and add 20 ml of 1×TTBS to the chamber with the membrane. Wash the membrane in 1×TTBS for 3×10 min in RT on agitation (30 rpm, 05 degrees angle). Prepare for development during the last washing step.

Development (Day 2)

During the last wash step, prepare the CLARITY Western ECL Substrate, 1:1 luminol enhancer: Peroxidase buffer, according to Table 9. For one membrane, add 3 ml of each solution to a 15 ml falcon tube (i.e. 6 ml substrate in total). Turn the falcon tube back and forth at least four times to mix the solution.

TABLE 9
Development solution preparation.

| | Amount of ClarityTM Western ECL Substrate | |
|---|---|---|
| Number of gels | (Luminol, Peroxidase) | |
| 2 | 12 ml | (6 ml, 6 ml) |
| 4 | 24 ml | (12 ml, 12 ml) |
| 6 | 36 ml | (18 ml, 18 ml) |
| 8 | 48 ml | (24 ml, 24 ml) |
| 12 | 72 ml | (36 ml, 36 ml) |

When the last wash is finished, pour off the 1×TTBS to a waste chamber. Pour the Clarity™ Western ECL Substrate from the falcon tube onto the membrane. Make sure that the entire membrane is covered with substrate by checking for dry spots. Incubate the membrane in the substrate for 3 min. While the membrane is being incubated, prepare for capturing an image of the membrane and ultimately image the membrane. In some aspects, the membrane is captured for a duration of 0.5-30 seconds.

### D. Densitometric Quantification of Gels and Analysis

In some aspects, the images obtained from the western blot are quantified using the software ImageLab. This is software developed for the usages together with the CCD camera, both from Bio-Rad. Using the ImageLab, the bands of proteins can be quantified depending on the intensity of each pixel in the bands. In this way a ratio may be obtained between the intensities of the bands showing uncleaved and cleaved SNAP25. ImageLab calculates total volume of the bands in each lane, which are manually separated. In some aspects, from the total volumes the software gives a Band % that is used for EC50 calculation with GraphPad Prism. The Band % can be used for effect calculations since 100% effect corresponds to 100% cleaved SNAP 25. The EC50 value is the concentration of the Galderma drug product at which half of the response is given and 50% of all SNAP25 is cleaved.

In some aspects, the images obtained of the membranes from the western blot (WB) procedure are analyzed in order to calculate an EC50 value for the Galderma drug product. The EC50 value corresponds to the concentration of the Galderma drug product that gives half of the response. In this case the EC50 value is decided as a 50/50 ratio between cleaved and uncleaved SNAP25. Hence, a densitometrical quantification of the protein bands from the WB is used to determine the ratios of the proteins in each sample from the cells treated with various concentrations of the toxin.

In some aspects, this protocol describes how to perform a densitometric quantification using the software Image Lab. The generated data will then be further analyzed in the software GraphPad Prism or any similar performing software.

In some aspects, prior to beginning analysis, ensure that there are no overexposed bands among the bands that are to be analyzed. The intensity percentages may not be correct if overexposed bands are included in the analysis. Overexposed bands will be highlighted in red by the software. In some aspects, if there are red highlights in the image, choose a new image with a shorter exposure time.

In some aspects, in Image Lab, click on the button “Image tools” in the Analysis Tool Box (the left column of the starting window). If necessary, flip the membrane vertically or horizontally by clicking the corresponding button under the section “Flip” in the left column (i.e. “Vertical” or “Horizontal”). The highest toxin concentration should be to the left, to easier keep track of which lane that corresponds to a certain column from the cell plate that was analyzed. It is also possible to rotate the picture by clicking on the button “Custom” under the section “Rotate”. The protein bands should be as horizontal as possible, to simplify the analysis. After clicking “Custom”, grab the red arrow that appeared on the image and drag it to the right or left to the desired rotation; right click on the image and chose “Rotate” to implement the rotation. Click “Ctrl+Z” on the keyboard to redo any unsatisfactory changes.

In some aspects, in Image Lab, return to the Analysis Tool Box by clicking on the arrow on the left hand side of the “Image Tools” heading at the top of the left column in the starting window. Click on the button “Lane and Bands” in the Analysis Tool Box. Under the section “Lane Finder”, chose “Manual . . . ”. In the new window that opened, type in the number of lanes that are to be analyzed. Resize the frame for the lanes by grabbing and dragging the corners or sides of the frame. The frame should be adjusted so that all bands that are to be detected are completely within the outer lines of the frame.

In some aspects, when all bands are within the outer lines of the frame, click the “Adjust background . . . ”-button in the left column of the starting window. In the new window that opened set the Disk Size (in the section “Background Subtraction” at the bottom of the window) to 1.0 mm and click “Apply to all lanes.”

In some aspects, when all bands are fitted into the boxes (two bands in each box, the upper for uncleaved SNAP25 and the lower for cleaved SNAP25), chose the tab “Bands” at the top of the left column in the starting window. Under the section “Band Finder” click on the button “Detect Bands . . . ”

In some aspects, check in the “Advance” button under the section “Detection Settings” and “Band Detection Sensitivity”. Set the Sensitivity to 80.00; Size Scale to 3; Noise Filter to 3, and Shoulder to 6. These settings alter the resolution at which the bands are being detected. Then click on the “Detect”-button at the bottom of the “Band Detection” window.

In some aspects, it may be beneficial to remove any unwanted detection bands, i.e. if there are two bands created for a single protein band or if there are bands outside of the two bands that are to be analyzed. This is done by clicking on the “Delete” button in the left column of the starting window and then clicking on the bands that are to be removed. Bands can also be added by using the “Add” button. In this case, the limits for the existing bands might need to be adjusted using the “Adjust” button, to make it possible to add a band where wanted.

In some aspects, it might be necessary to adjust the limits of each detection band to ensure that the protein bands are quantified in the most correct manner. At the top of the starting window, click on the button “Lane Profile”. A new window will appear. Make sure that you have unmarked the “Detect”, “Add” or “Adjust” buttons from the previous step by clicking these icons again. Then click on the first lane box. In the “Lane Profile” window, the intensity of the protein bands is now shown as a curve and it is possible to adjust the limits of the detection bands accordingly. Click on the blue lines beneath the curve plot and drag these sideways to adjust the limits. Make sure that all tops in the curve are fully within the limits, and following these guidelines:

Do not adjust the limits if not necessary. When there are two separate tops, make sure that the smaller top is fully within the limit if the default gap between the band limits does not fit between the curves. If the curves are not completely separated, adjust the limit of the smaller bulb so that the detection band is directly above the protein band. If a double band was removed, the limit of the band might be in the middle of the curve. Then adjust the limit to fit the entire curve, i.e. until the curve meets the background line.

When the limits have been satisfactorily set, the “Lane Profile” window may be closed. To retrieve the quantification of the bands click on the button “Analysis Table” in the top bar of the starting window. The “Analysis Table” will appear at the bottom of the window. At the top of the “Analysis Table”, click on the button for changing the orientation of the analysis table, so that the data for each lane is shown on top of each other, as a vertical column instead of a horizontal line as is set as default. Then click on the button “Export analysis table to Excel” at the top of the “Analysis Table”. The band data will now be opened in an Excel file.

In the Excel data file there will now be one or two rows for each lane from the image analysis, one row for each protein band. If there are two rows, i.e. two bands, with the same lane number, the band number 1 will be for the uncleaved SNAP25 and band number 2 for the cleaved SNAP25. In the Excel file, copy the Band % for the cleaved bands to a new column to more easily copy the data for further analysis. Start by naming one column “Sample” and write down each sample that was analyzed, i.e. if the rows B and C from one plate were included on this image, name the rows B10, B9, B8, . . . , B1, C5, C4, . . . , C1. Then name the column next to the “Sample” column “% Cleaved” and copy the Band % for each sample to the corresponding row.

In some aspects, in the instance of only one band in the gel, go back to the image and check if it's a 100% cleaved or uncleaved band, and add 100 or 0 accordingly. The sample data are preferably added from low to high toxin concentration, to ease the transfer of the data to GraphPad Prism for analysis.

EC₅₀ Analysis with GraphPad Prism (v. 5.02)

In some aspects, the EC₅₀ is determined utilizing GraphPad Prism to visualize the data, as follows:

In some aspects, in order to analyze the data sets, click on Analyze and under XY analyses choose Nonlinear regression (curve fit), thereafter OK.

In some aspects, choose under Dose-response—Stimulation, Log(agonist) vs response—Variable slope (four parameters). Thereafter, click on the tab Constrain and choose Constant equal to 0 for bottom and for top Constant equal to 100. This will result in a constrained analysis. Click OK.

In some aspects, this will result in a table with absolute EC₅₀ values including standard Error. Relative potencies are calculated by dividing the EC₅₀ values for Test and QC samples with the reference sample. The standard error for the relative potency is calculated based on error propagation according to:

SE RP(QC)=RP(QC)*SQRT((SE Ref/EC₅₀Ref)+(SE QC/EC₅₀QC)²)

SE RP(Test)=RP(Test)*SQRT((SE Ref/EC₅₀Ref)+(SE Test/EC₅₀Test))

Where SE is standard error and RP is relative potency. Note, this calculation for relative potency is valid only if the three curves are parallel.

The graph is plotted automatically, and can be found under the Graphs sections in the left column. The X-axis may be formatted to go from −5 to +2 on the log scale while the Y axis may be formatted to 110-150, depending on the needs evidenced by the data.

EC₅₀ Analysis with R (v. 3.6.1)

In some aspects, the EC₅₀ analysis is performed with R through the dose response curves (DRC) package (3.0-1). See Ritz et al. (PLoS One. 2015 Dec. 30; 10(12):e0146021. doi: 10.1371/journal.pone.0146021).

For DRC, a table of a specific format must be created that provides the raw data captured from the densitometry images of the experimental gels.

Start the R environment. At the prompt, enter the following:

library(drc) (This loads the DRC package)

setwd(“C:/DRC”) (This sets working directory to DRC)

source(“3plate.rs”) (This executes the script which performs the analysis)

In some aspects, the 3plate.rs script utilized in the analysis is provided in FIG. 8A-8B.

### E. Statistical Analysis

In some aspects, statistical analysis of the data is performed with GraphPad Prism (v. 5.0). In some aspects, a 4 parametric logistic (4PL) curve is fitted to the data using a model constraining the curve between 0 and 100%. The data resulting from experiments 8 and 4 (discussed in the examples section) were analyzed with GraphPad Prism.

In some aspects, statistical analysis of the data is performed in R (v. 3.6.1, 64 bit) utilizing methods implemented in the dose response curves (DRC) package (3.0-1). In some aspects, a 4PL curve is fitted to the data using an unconstrained model. In some aspects, errors are expressed as standard errors and error propagation may be used to estimate the error for relative potencies. In some aspects, the R statistical analysis may use the script detailed in FIG. 7A-7B or FIG. 8A-8B.

### EXAMPLES

### Example 1

### Investigation of Linearity

Purpose: to investigate whether the Western blotting quantification is linear at different protein concentrations and in different ranges in the dilution series.

In order to optimize the quantification of the Western blot, the analysis was run on samples with known concentrations of cleaved and uncleaved SNAP25. The samples for this analysis were prepared by serial diluting protein samples from cells with 30 U/ml BoNT/A—treatment with protein samples from cells added only DP-buffer. Protein samples from the toxin-treated cells were expected to contain 100% cleaved SNAP25 and the samples from non-treated cells 100% uncleaved SNAP25. However, when analyzing the undiluted protein samples it was concluded that the protein samples from the toxin-treated cells had only 95% cleaved SNAP25. The final concentrations in the serial dilution series after adjusting according to the incomplete cleavage of SNAP25 is shown in Table 10.

TABLE 10
Final concentrations in serial diluted protein samples,
from toxin-treated and DP-buffer added cells.

| Sample | Concentration of Cleaved SNAP25 (%) |
|---|---|
| W1 | 0 |
| C6 | 2.9685 |
| C5 | 5.9375 |
| C4 | 11.875 |
| C3 | 23.75 |
| C2 | 47.5 |
| W2 | 47.5 |
| W3 | 71.275 |
| W4 | 83.1375 |
| W5 | 89.06875 |
| W6 | 92.03475 |
| C1 | 95 |

The protein dilution series were analyzed using Western blot, as described herein. Six gels were run, with 1 μl, 2 μl, 3 μl, 4 μl, 5 μl, and 6 μl of protein sample, respectively. To evaluate the precision and accuracy of the western blot the RSD and absolute error was calculated between the runs. The analysis was performed using both ImageLab (Table 11) and ImageJ (Table 12). Further, the measured % cleaved was plotted against the expected values of % cleaved, to investigate the linearity (FIG. 10). It was found that the samples with low and high concentration of cleaved SNAP25 appeared to be underestimated with the western blot analysis. Therefore, the average Adj. Band Vol. for cleaved and uncleaved SNAP25 was plotted against the expected values of each (FIG. 9). This was to evaluate if the underestimation of the % cleaved was depending on the band intensities of the cleaved protein or the uncleaved. The result indicated that the trend of underestimation for high and low percent of toxin-treated cells appeared when analyzing the bands with cleaved SNAP25.

TABLE 11
Precision and accuracy of the Western blot
run on protein dilution series, using ImageLab.
ImageLab

| | | | | Expected % | True % | Absolute |
|---|---|---|---|---|---|---|
| Sample | Average | STDev | RSD | Cleaved | Cleaved | error |
| W1 | 0.000 | 0.000 | 0.000 | 0 | 0 | 0.000 |
| W2 | 62.096 | 3.132 | 5.044 | 50 | 47.5 | −14.596 |
| W3 | 83.035 | 2.960 | 3.565 | 75 | 71.275 | −11.760 |
| W4 | 89.662 | 2.652 | 2.958 | 87.5 | 83.138 | −6.524 |
| W5 | 92.773 | 2.077 | 2.239 | 93.75 | 89.069 | −3.704 |
| W6 | 93.462 | 1.847 | 1.976 | 96.875 | 92.035 | −1.427 |
| C1 | 95.073 | 1.927 | 2.027 | 100 | 95 | −0.073 |
| C2 | 68.636 | 4.567 | 6.654 | 50 | 47.5 | −21.136 |
| C3 | 38.538 | 3.009 | 7.809 | 25 | 23.75 | −14.788 |
| C4 | 15.024 | 2.690 | 17.903 | 12.5 | 11.875 | −3.149 |
| C5 | 3.936 | 1.188 | 30.184 | 6.25 | 5.938 | 2.001 |
| C6 | 11.642 | 2.757 | 23.680 | 3.125 | 2.969 | −8.673 |
| | Average | 2.401 | 8.670 | | Average | −6.986 |

TABLE 12
Precision and accuracy of the Western blot
run on protein dilution series, using ImageJ.
ImageJ

| | | | | Expected % | True % | Absolute |
|---|---|---|---|---|---|---|
| Sample | Average | STDev | RSD | Cleaved | Cleaved | error |
| W1 | 0.000 | 0.000 | 0.000 | 0 | 0 | 0.000 |
| W2 | 60.115 | 3.816 | 6.348 | 50 | 47.5 | −12.615 |
| W3 | 81.206 | 3.421 | 4.213 | 75 | 71.275 | −9.931 |
| W4 | 88.306 | 2.316 | 2.623 | 87.5 | 83.138 | −5.168 |
| W5 | 91.584 | 1.972 | 2.154 | 93.75 | 89.069 | −2.515 |
| W6 | 92.800 | 1.610 | 1.735 | 96.875 | 92.035 | −0.765 |
| C1 | 94.104 | 1.087 | 1.155 | 100 | 95 | 0.896 |
| C2 | 67.715 | 3.624 | 5.351 | 50 | 47.5 | −20.215 |
| C3 | 35.870 | 4.098 | 11.424 | 25 | 23.75 | −12.120 |
| C4 | 14.081 | 2.226 | 15.808 | 12.5 | 11.875 | −2.206 |
| C5 | 4.078 | 0.801 | 19.642 | 6.25 | 5.938 | 1.859 |
| C6 | 0.649 | 0.359 | 55.358 | 3.125 | 2.969 | 2.319 |
| | Average | 2.111 | 10.484 | | Average | −5.038 |

### Example 2

### Investigation of an Apparent Increase of Total SNAP25 in Response to BoNT/A

Purpose: It was observed that total SNAP25 seems to vary with toxin concentration; the purpose was to investigate if this is a systematic effect.

When examining the Western blot images it was suggested that there was a difference in band intensity between the protein samples from motor neurons treated with high concentration of toxin and low or no concentration of toxin. In order to evaluate this, the total band intensities were analyzed instead of the percent of the band with only cleaved SNAP25. For this, all data from experiments 10, 12, 14, 16 and 18 was used. This was done by exporting the “Lane Statistics-table” to Excel from the “Analysis table” in ImageLab for each membrane investigated and using the “Adj. Total Band Vol. (Int)” for analysis. No changes were done in the bands or lanes from the first analysis of % cleaved. The total band volume of each column corresponding to a level of toxin-treatment was then plotted against the toxin concentration, with 1=DP control and 10=30 U toxin treatment. This was done for experiments 10, 12, 14, 16 and 18, with and without gel outliers (FIG. 10). The result was also plotted in GraphPad Prism 5. To further evaluate the difference in the band intensities, the total band volumes were compared using a Student t-test in Excel. No statistically significant difference in band volumes was found between high and no toxin treatment. However, there was a statistically significant difference between high and no toxin-treatment compared to the columns with medium toxin concentration.

The found difference in band intensity was suggested to be due to a difference in longevity between the cells with medium toxin-treatment and high or no toxin-treatment. To evaluate this, the same analysis as described above was performed on the protein dilution series created using only protein samples from motor neurons treated with high toxin concentration or no toxin. These two samples were believed to have similar protein concentration and if no difference in band volume were found between high or no toxin and the medium high toxin, the significant difference in the previous analysis would be due to the cell longevity. However, the same result was found in this analysis, suggesting that the difference in band intensity would be due to the analysis rather than the protein samples. Further, this analysis was made in both ImageLab (FIG. 12) and ImageJ (FIG. 13), to compare the analysis of the two programs.

### Example 3

### Summary of Relative Potencies

Summary of the relative and absolute potencies obtained in experiments 4, 8, 10, 12, 14, 16, 18, and 20 for the different samples. The data is relative to the reference sample. Data and experimental details are drawn from the subsequent examples below.

TABLE 13
Relative potencies for all samples from experiments
4, 8, 10, 12, 14, 16, 18, and 20.

| Ex- | | | | Relative | Ex- | |
|---|---|---|---|---|---|---|
| peri- | | | | potency | pect- | Pre- |
| ment | | | | in cell | ed | ci- |
| num- | Date of | | | method | Potency | sion |
| ber | Assay | Sample | | (U/ml) | (U/ml) | (%) |
| 4 | | | | | | |
| 8 | 2019 Jun. 3 | | | | | |
| 10 | 2019 Sep. 2 | 16908 | (QC) | 89 ± 8.2 | 94 | 95 |
| 10 | 2019 Sep. 2 | 16767 | (Test) | 98 ± 9.3 | 100 | 98* |
| 12 | 2019 Sep. 16 | 16908 | (QC) | 85 ± 4.4 | 94 | 90 |
| 12 | 2019 Sep. 16 | 16767 | (Test) | 96 ± 4.6 | 100 | 96* |
| 14 | 2019 Sep. 23 | 16908 | (QC) | 83 ± 4.5 | 94 | 88 |
| 14 | 2019 Sep. 23 | 16767 | (Test) | 102 ± 5.4 | 100 | 98* |
| 16 | 2019 Sep. 30 | 16908 | (QC) | 83 ± 3.7 | 94 | 88 |
| 16 | 2019 Sep. 30 | 16997 | (Test) | 98 ± 5.0 | 79 | 80 |
| 18 | 2019 Nov. 4 | 16908 | (QC) | 99 ± 5.1 | 77 | 77 |
| 18 | 2019 Nov. 4 | 16908 | (Test)* | 110 ± 5.0 | 94 | 80 |
| 20 | 2019 Nov. 11 | 17235 | (QC) | 102 ± 4.4 | 108 | 94 |
| 20 | 2019 Nov. 11 | 16997 | (Test) | 95 ± 4.6 | 79 | 83 |

All values are against sample 16767 as reference.

From the data presented in Table 13 we can see that sample 16908 shows a reduction in potency over time, making it unsuitable for calculating precision and assessing linearity with LD₅₀. Also, sample 16997 is unsuitable because it is likely to have a much higher potency then the 77 U/ml that was obtained with LD₅₀. Sample 17235 is likely to have an accurate determination of 108 U/ml, although the 16767 sample has degraded most likely showing an artificially low LD₅₀ for 17235. We therefore only use the reference vs reference, marked with * in the table above (Experiments 10, 12 and 16) to calculate precision. Here we get 98%, 96%, and 98% respectively, giving an average precision of 97.5%, i.e. a deviation of 2.5% from target.

Table 14 below shows the average % RSD for each curve in the different experiments.

TABLE 14
% for all samples from experiments
4, 8, 10, 12, 14, 16, 18, and 20.

| | % RSD | % RSD | % RSD |
|---|---|---|---|
| Experiment | (Ref) | (QC) | (Test) |
| 10 | 19.188 | 17.451 | 17.633 |
| 12 | 16.562 | 17.205 | 13.812 |
| 14 | 7.269 | 9.589 | 16.574 |
| 16 | 31.386 | 21.412 | 25.829 |
| 18 | 11.131 | 9.154 | 16.331 |
| 20 | 12.752 | 12.955 | 12.858 |

All values are against sample 16767 as reference.

Most % RSDs are below 20% but in general higher than expected. This is because there is for most curves in the high toxin data points weak bands of un-cleaved toxin in one or two wells (about 5% un-cleaved, 95% cleaved), while the other points all have no detectable un-cleaved material, resulting in 0% un-cleaved. This results in very high variability and high % RSD. Also variation in the low end of the dilution series is large, measured in percentage (i.e between 3 and 6% cleaved, giving a variability of 100%). We have preliminary data suggesting that this problem is due to the quantification with ImageLab software and seems to be reduced or completely mitigated by using ImageJ for quantification.

Developmental experimentation generally relied on relative potencies, but absolute potencies can also be used to assess stability of the method. In Table 15 we present absolute potencies as obtained by the EC₅₀ value, expressed as U/ml.

TABLE 15
Absolute potencies for all samples from experiments 4, 8, 10, 12, 14, 16, 18, and 20.

| Experiment | Date of | 16767 | 16908 | 16997 | 16908* | 17235 |
|---|---|---|---|---|---|---|
| number | Assay | (U/ml) | (U/ml) | (U/ml) | (U/ml) | (U/ml) |
| 4 | | | | | | |
| 8 | 2019 Jun. 3 | | | | | |
| 10 | 2019 Sep. 2 | 2.27 ± 0.16 | 2.54 ± 0.15 | | | |
| 10 | 2019 Sep. 2 | 2.32 ± 0.16 | | | | |
| 12 | 2019 Sep. 16 | 2.45 ± 0.08 | 2.39 ± 0.07 | | | |
| 12 | 2019 Sep. 16 | 2.54 ± 0.09 | | | | |
| 14 | 2019 Sep. 23 | 1.91 ± 0.06 | 2.3 ± 0.10 | | | |
| 14 | 2019 Sep. 23 | 1.87 ± 0.08 | | | | |
| 16 | 2019 Sep. 30 | 2.05 ± 0.07 | 2.47 ± 0.07 | 2.1 ± 0.08 | | |
| 18 | 2019 Nov. 4 | 2.62 ± 0.09 | 2.64 ± 0.10 | | 2.38 ± 0.07 | |
| 20 | 2019 Nov. 11 | 2.45 ± 0.08 | | 2.57 ± 0.09 | | 2.41 ± 0.07 |

It is evident that the absolute potencies varies more than the relative potencies (for example, from 1.87 to 2.64 for 16767), suggesting that absolute values may not be feasible with the current assay. The variability is most likely due to differences in cell culturing conditions.

### Example 4

Measurement of potency of a degraded Galderma drug product sample suggest that the method is stability indicating.

This example demonstrates a trend for sample 16908, which was used as a QC sample in the assays. The data is collected from experiments 8, 10, 12, 14, 16 and compared with the relative potencies obtained from the same sample in LD₅₀ assays. All potencies (Table 16) from the cell method are relative to sample 16761, which was used as a reference, assuming a potency of the reference at 98 U/ml.

TABLE 16
Relative potencies for sample 16908 at different dates obtained
with the cell based method compared to LD₅₀ data.

| | Potency in cell method | Potency in LD₅₀ |
|---|---|---|
| Date of Assay | (U/ml) | (U/ml) |
| 2019 Feb. 18 | | 94 ± 8 |
| 2019 Feb. 18* | | 100 ± 9 |
| 2019 Jun. 3 | 96 ± ? | |
| 2019 Aug. 18* | | 77 ± 6 |
| 2019 Sep. 2 | 89 ± 8.2 | |
| 2019 Sep. 16 | 85 ± 4.4 | |
| 2019 Sep. 23 | 83 ± 4.5 | |
| 2019 Sep. 30 | 83 ± 3.7 | |

All samples were stored at 5° C.
Dates marked with * are calculated 3 and 6 months from start date, and are approximations.

FIG. 15 depicts this data in a line graph, with LD₅₀ data in circular data points and cell based data in square data points. Solid lines are actual data and dotted lines are lines obtained with linear regression. The R² values are 0.48 for the cell based data and 0.51 for the LD₅₀ data, suggesting the methods are linear, suggesting that the cell based method is parallel with LD₅₀.

### Example 5

### Experiment 8

Purpose: To investigate the differences between two batches of motor neurons, and old batch and a new batch.

One vial of each batch of motor neurons was cultivated onto two 96-well plates each. Cells from the old lot were enough for plating one full plate (plate 1) and one plate (plate 2) with nine full columns+three wells in the last column 11. Reference sample was 16761 (98 U/mL). Assay control sample (QC) was 16908 (94 U/mL).

The new batch of cells were enough for plating one full plate (plate 1; inner 60 wells) and one plate (plate 2) nine full columns+four wells in the last column 11. The cells in plate 1 for both lots were cultivated in medium from Cellular Dynamics. The cells in plate 2 for both lots were first cultivated in medium from Cellular Dynamics but were, at every media change, replaced with 50% BRAINPHYS Neuronal Medium from StemCell Technologies. There were no differences between the two batches of motor neurons.

### Example 6

### Experiment 10

Purpose: To evaluated three different methods of toxin dilution.

TO investigate the difference in variance between the three pipetting methods, the % RSD was calculated per sample per plate, the results of which are presented in Table 17. Plate 1 samples were serially diluted via automatic multichannel pipette. Plate 2 samples were serially diluted via manual multichannel pipette. Plate 3 samples were serially diluted via manual single channel pipette.

Reference sample was 16761 (98 U/mL). Assay control sample (QC) was 16908 (94 U/mL). Unknown sample was 16761 (98 U/mL).

TABLE 17
Summary of potencies for each plate.

| | Plate 1 | Plate 2 | Plate 3 |
|---|---|---|---|
| Sample | (% RSD) | (% RSD) | (% RSD) |
| Reference | 27.3 | 12.0 | 22.0 |
| QC | 13.3 | 28.3 | 11.7 |
| Test | 24.9 | 33.3 | 7.7 |

Errors are expressed as standard errors.

The EC₅₀ and relative potencies were calculated for each plate and for each curve, to assess the effect of the different dilution methods, and the data for which is presented in Table 18.

TABLE 18
Summary of potencies for each plate.

| | Absolute Potency | Relative Potency |
|---|---|---|
| Sample | (EC₅₀ U/mL) | (% of Reference U/mL |
| Reference (Plate 1) | 2.29 ± 0.29 | N/A |
| QC (Plate 1) | 2.64 ± 0.28 | 87 ± 14 |
| Test (Plate 1) | 1.98 ± 0.16 | 116 ± 14 |
| Reference (Plate 2) | 2.02 ± 0.22 | N/A |
| QC (Plate 2) | 2.07 ± 0.21 | 98 ± 14 |
| Test (Plate 2) | 1.75 ± 0.18 | 115 ± 17 |
| Reference (Plate 2) | 2.42 ± 0.33 | N/A |
| QC (Plate 3) | 2.87 ± 0.25 | 84 ± 14 |
| Test (Plate 3) | 3.07 ± 0.35 | 79 ± 14 |

Errors are expressed as standard errors.

A calculation of EC₅₀ based on all three plates was performed according to the methods described herein, even though different pipetting methods were used for the dilution of toxin in the three plates. This is reasonable, because the difference between the methods were minimal, see Table 19.

TABLE 19
Summary of potencies.

| | Absolute Potency | Relative Potency | Average |
|---|---|---|---|
| Sample | (EC₅₀ U/mL) | (% of Reference U/mL | % RSD |
| Reference | 2.27 ± 0.16 | N/A | 21.8 |
| QC | 2.54 ± 0.15 | 89 ± 8.2 | 21.1 |
| Test | 2.32 ± 0.16 | 98 ± 9.6 | 22.6 |

Errors are expressed as standard errors.

Cell Culture and Toxin Treatment

Geltrex (#A1413301, ThermoFisher) diluted in Neurobasal medium (#12348017, ThermoFisher) was used for coating. After coating, plates were incubated for 50 min at 37° C. and thereafter 50 min at room-temperature. Two vials of Motor Neurons were seeded on three 96-well plates (inner 60 well). The cells were mixed together after centrifugation and dilution in media. The cell suspension was diluted up to 36 mL. Last column (column 11) got <200 uL cell suspension added to the wells. Cells were treated on DIC 12 with the Galderma drug product.

Western Blot

Bio-Rad ladder (161-0374) was used. Only anti-SNAP25 primary antibody (S9684) was used. The running buffer and 1×TBS were measured out using the approximate volumes graded on the 1000 ml glass flask. The tween-20 was pipetted using Pasteur pipet. The ECL-substrate was reused between the membranes.

Sample 6 from row E on the plate with manual pipet appeared to have a larger volume when loaded into well for electrophoresis.

Dilution with automatic multichannel pipette was the most suitable method when taking into account variance, and this is the method utilized in the proceeding experiments.

### Example 7

### Experiment 12

Purpose: To investigate the accuracy and precision in the cell based potency method by comparing the outcome of the reference and the unknown when these are the same sample (i.e. reference vs reference).

Reference sample was 16761 (98 U/mL). Assay control sample (QC) was 16908 (94 U/mL). Unknown sample was 16761 (98 U/mL). The results of the assay are depicted in Table 20.

TABLE 20
Summary of potencies.

| | Absolute Potency | Relative Potency |
|---|---|---|
| Sample | (EC₅₀ U/mL) | (% of Reference U/mL |
| Reference | 2.45 ± 0.08 | N/A |
| QC | 2.39 ± 0.07 | 85 ± 4.4 |
| Unknown | 2.54 ± 0.09 | 96 ± 4.6 |

Errors are expressed as standard errors.

Cell Culture and Toxin Treatment

Two vials of Motor Neurons were seeded on three 96-well plates (inner 60 well). The cells were mixed together after centrifugation and dilution in media. The cell suspension was diluted up to 38 mL. Cells were treated on DIC 12 with the Galderma drug product.

Western Blot

Bio-Rad ladder (161-0374) was used. Only anti-SNAP25 primary antibody (S9684) was used. The running buffer and 1×TBS were measured out using the approximate volumes graded on the 1000 ml glass flask. The tween-20 was pipetted using Pasteur pipet. The ECL-substrate was reused between the membranes.

The difference between the reference and the unknown might be due to the difference in the intensity of the bands that were observed. The reason for the intensity difference could be insufficient mixing of the protein samples before sample preparation.

### Example 8

### Experiment 14

Purpose: To investigate the accuracy and precision in the cell based potency method by comparing the outcome of the reference and the unknown when these are the same sample (i.e. reference vs. reference).

Reference sample was 16761 (98 U/mL). Assay control sample (QC) was 16908 (94 U/mL). Unknown sample was 16761 (98 U/mL). The results of the assay are depicted in Table 21.

TABLE 21
Summary of potencies.

| | Absolute Potency | Relative Potency | Average |
|---|---|---|---|
| Sample | (EC₅₀ U/mL) | (% of Reference U/mL | % RSD |
| Reference | 1.91 ± 0.06 | N/A | |
| QC | 2.3 ± 0.10 | 83 ± 4.5 | |
| Unknown | 1.87 ± 0.08 | 102 ± 5.4 | |

Errors are expressed as standard errors.

Cell Culture and Toxin Treatment

Two vials of Motor Neurons were seeded on three 96-well plates (inner 60 well). The cells were mixed together after centrifugation and dilution in media. The cell suspension was diluted up to 38 mL. Cells were treated on DIC 12 with the Galderma drug product.

No ladder was used, in order to use all wells for protein samples. Only anti-SNAP25 primary antibody (S9684) was used. The running buffer and 1×TBS were measured out using the approximate volumes graded on the 1000 ml glass flask. The tween-20 was pipetted using Pasteur pipet. The ECL-substrate was reused between the membranes.

Running 15 samples per gel without ladder works well, and this is utilized in the proceeding experiments. Running reference as test gives a deviation of 2% from expected and QC (16908) is lower than expected.

### Example 9

### Experiment 16

Purpose: To compare the outcomes of the cell based potency method between two toxin units from the same DS batch, namely 79 and 98 U/mL.

Reference sample was 16761 (98 U/mL). Assay control sample (QC) was 16908 (94 U/mL). Unknown sample was 16997 (79 U/mL). The results of the assay are depicted in Table 22.

TABLE 22
Summary of potencies.

| | Absolute Potency | Relative Potency | Average |
|---|---|---|---|
| Sample | (EC₅₀ U/mL) | (% of Reference U/mL | % RSD |
| Reference | 2.05 ± 0.07 | N/A | |
| QC | 2.47 ± 0.07 | 83 ± 3.7 | |
| Unknown | 2.1 ± 0.08 | 98 ± 5.0 | |

Errors are expressed as standard errors.

Cell Culture and Toxin Treatment

Two vials of Motor Neurons were seeded on three 96-well plates (inner 60 well). The cells were mixed together after centrifugation and dilution in media. The cell suspension was diluted up to 38 mL. Cells were treated on DIC 12 with the Galderma drug product.

Western Blot

No ladder was used, in order to use all wells for protein samples. Only anti-SNAP25 primary antibody (S9684) was used. The ECL-substrate was reused between some of the membranes.

The relative potency of the 79 U/ml toxin (16997) was 20% higher than expected, suggesting that there has been a drop in the potency of the 98 U/ml toxin (16767) reference, or that the 16997 sample has a higher potency than the initial LD₅₀ value suggests.

### Example 10

### Experiment 18

Purpose: To compare the absolute potencies of one 94 U/mL toxin sample stored in the fridge from day 0 and one stored in the freezer for approximately five months prior to the potency assay (stored at −80° C.).

Reference sample was 16761 (98 U/mL). Assay control sample (QC) was 16908 (94 U/mL, stored in fridge). Unknown sample was 16908 (94 U/mL, stored in freezer). The results of the assay are depicted in Table 23.

TABLE 23
Summary of potencies.

| | Absolute Potency | Relative Potency | Average |
|---|---|---|---|
| Sample | (EC₅₀ U/mL) | (% of Reference U/mL | % RSD |
| Reference | 2.62 ± 0.09 | N/A | |
| QC | 2.64 ± 0.10 | 99 ± 5.1 | |
| Unknown | 2.38 ± 0.07 | 110 ± 5.0 | |

Errors are expressed as standard errors.

Cell Culture and Toxin Treatment

Two vials of Motor Neurons were seeded on three 96-well plates (inner 60 well). The cells were mixed together after centrifugation and dilution in media. The cell suspension was diluted up to 38 mL. Cells were treated on DIC 12 with the Galderma drug product.

Western Blot

No ladder was used, in order to use all wells for protein samples. Only anti-SNAP25 primary antibody (S9684) was used.

There was an 11% difference in absolute potency between the 94 U/mL samples. This suggests that the 94 U/mL sample stored in the refrigerator had an 11% potency loss during the months in the refrigerator from when the other sample was frozen.

### Example 11

### Experiment 20

Purpose: To compare the potencies of the 108 U/mL toxin and the 79 and 98 U/mL toxins in order to investigate any loss in potency of the latter two.

Reference sample was 16761 (98 U/mL). Assay control sample (QC) was 17235 (108 U/mL). Unknown sample was 16997 (79 U/mL). The results of the assay are depicted in Table 24.

TABLE 24
Summary of potencies.

| | Absolute Potency | Relative Potency | Average |
|---|---|---|---|
| Sample | (EC₅₀ U/mL) | (% of Reference U/mL | % RSD |
| Reference | 2.45 ± 0.08 | N/A | |
| QC | 2.41 ± 0.07 | 102 ± 4.4 | |
| Unknown | 2.57 ± 0.09 | 95 ± 4.6 | |

Errors are expressed as standard errors.

Cell Culture and Toxin Treatment

The PDL and the Geltrex Matrix were incubated for approximately 1 hour each. Both were aspirated using vacuum suction.

The supplements and the cell media were left in room temperature for approximately 1 hour before thawing and plating of motor neurons. Two vials of cells were used, to suffice for three plates. The cells were not counted. 200 μl of cell suspension was added to each well. The Geltrex matrix was aspirated five columns at the time from the cell plate and the cell suspension added accordingly, using a multi-dispensing electric pipet.

On day 2, 5 and 7 after seeding 75% of the media was exchanged for new Complete Maintenance media+DAPT. On day 9 after seeding 35% of the media was exchanged again with Complete Maintenance media+DAPT. This was to make sure that the media would suffice for the last media exchange, when toxing the cells.

The cells were toxin treated 12 days after seeding. One cell plate was treated at the time. 130 μl of cell media was aspirated from each culture vessel in the cell plate. 12 ml of new complete maintenance media mixed with a few mL of complete maintenance media+DAPT, was added to the aspirated portion. 479.2 μl of the media was then added to the first column of the mixing plate and 300 μl to column 2-9. Column 10 was left empty as this column of cells would serve as a DP buffer control.

The toxin was added to the first column of the mixing plate by pipetting against the wall of the wells.

Western Blot

No ladder was used, in order to use all wells for protein samples. Only anti-SNAP25 primary antibody (S9684) was used.

17235 has a relative potency of 102±4.4 U/ml with 16767 as a reference. 16997 is again higher than expected at 95±4.6, and has lost about 8 U over the course of −1 month. The data suggest that the reference sample is about 5 U/ml too low (based on the previous measure of 16997 which gave 83 U). This would put 17235 at 110 U, close to expected. However, these differences are within the standard error of the method.

### Example 12

Purpose: To compare the toxin potency determination assay of the Galderma drug product and three commercial BoNT/A products; DYNASPORT, BOTOX, and XEOMIN. The commercial products were dissolved to a concentration of 100 U according to the respective product inserts.

TABLE 25
Relative potencies of the BoNT/A products.

| | Relative potency to | |
|---|---|---|
| | Galderma Drug Product | Parallelism |
| Product | (U/ml) | against reference |
| QC | 105 ± 3.9 | 1.02 |
| Galderma Drug Product | 99 ± 3.6 | 1.04 |
| DYSPORT | 97 ± 3.6 | 1.05 |
| BOTOX | 116 ± 4.3 | 0.96 |
| XEOMIN | 114 ± 4.2 | 1.01 |

In Table 25, relative potency is expressed in Galderma drug product (U/ml) and parallelisms as ratio of Hill slope. The expected values are 98 U/ml for QC, 101 U/ml for Galderma drug product, and 100 U/ml for the three commercial toxins, and the expected value for the parallelism assessment is 1.0. The data demonstrates that the methods described herein can be used to assess several different BoNT/A products which are bioequivalent to one another in the assay, based on the high degree of parallelism. See FIG. 16A-16D.

One of ordinary skill in the art would recognize that the ability to simply draw direct comparisons of potency between different products is surprising and unexpected result given that this cannot be done with the industry standard that relies on LD₅₀ experiments in mice.

The methods illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof. It is recognized that various modifications are possible within the scope of the disclosure claimed. Thus, it should be understood that although the present disclosure has been specifically disclosed by preferred embodiments and optional features, modification and variation of the disclosure embodied therein herein disclosed may be resorted to by those skilled in the art, and that such modifications and variations are considered to be within the scope of this disclosure.

The disclosure has been described broadly and generically herein. Each of the narrower species and subgeneric groupings falling within the generic disclosure also form part of the methods. This includes the generic description of the methods with a proviso or negative limitation removing any subject matter from the genus, regardless of whether or not the excised material is specifically recited herein. The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

One skilled in the art readily appreciates that the present disclosure is well adapted to carry out the objects and obtain the ends and advantages mentioned, as well as those inherent therein. Modifications therein and other uses will occur to those skilled in the art. These modifications are encompassed within the spirit of the disclosure and are defined by the scope of the claims, which set forth non-limiting embodiments of the disclosure.

In addition, where features or aspects of the disclosure are described in terms of Markush groups, those skilled in the art will recognize that the disclosure is also thereby described in terms of any individual member or subgroup of members of the Markush group.

All references, articles, publications, patents, patent publications, and patent applications cited herein are incorporated by reference in their entireties for all purposes.

However, mention of any reference, article, publication, patent, patent publication, and patent application cited herein is not, and should not be taken as, an acknowledgment or any form of suggestion that they constitute valid prior art or form part of the common general knowledge in any country in the world.

What is claimed:

1. A method of determining potency of botulinum neurotoxins (BoNTs), the method comprising:
(a) distributing at least two different BoNT samples to at least two containers comprising cells expressing a SNAP25 protein, wherein the first BoNT sample is a reference sample of a known potency and the second BoNT sample is a test sample of unknown potency,
(b) incubating the cells with the BoNT for a period of time,
(c) determining the ratio of cleaved SNAP25 protein to uncleaved SNAP25 protein corresponding to the reference sample and the test sample, and
(d) identifying the potency of the test sample relative to the reference sample.

2. The method according to claim 1, wherein a third BoNT sample, a quality control sample, of known potency, is distributed to a third container and utilized as a positive control.

3. The method of claim 1, wherein the relative potency of the test sample is at least 95% as accurate as compared to a murine LD₅₀ assay.

4. The method of claim 1, wherein (c) comprises subjecting the cleaved and uncleaved SNAP25 proteins to Western blot and densitometric quantification.

5. The method according to claim 1, wherein the period of time is for at least 6, 12, 16, 20, 24, 32, 40, 48, or 56 hours.

6. The method according to claim 1, wherein the at least two containers each comprise a plurality of wells.

7. The method according to claim 6, wherein the at least two different BoNT samples are serially diluted across the plurality of wells.

8. The method according to claim 1, wherein the at least two containers are tissue culture plates.

9. The method according to claim 1, wherein the at least two containers are 48-, 96-, 384-, or 1536-well plates.

10. The method according to claim 1, wherein the cells are adhered or attached to the at least two containers.

11. The method according to claim 1, wherein the cells natively express SNAP25.

12. The method according to claim 1, wherein the cells express a heterologous SNAP25.

13. The method according to claim 1, wherein the cells are non-neuronal cells.

14. The method according to claim 1, wherein the cells are genetically modified.

15. The method according to claim 1, wherein the cells are neuronal cells.

16. The method according to claim 15, wherein the neuronal cells are motor neurons.

17. The method according to claim 1, wherein the cells are treated with a non-proliferation agent.

18. The method according to claim 17, wherein the non-proliferation agent inhibits γ-secretase.

19. The method according to claim 17, wherein the non-proliferation agent is DAPT.

20. The method according to claim 1, wherein a protease inhibitor is added to the at least two containers upon conclusion of (b).

21. The method according to claim 1, wherein the cells are lysed after incubating the cells with the BoNT.

22. The method according to claim 21, wherein the cells are lysed by sonication.

23. The method according to claim 21, wherein the cells are lysed by addition of a lysis agent.

24. The method according to claim 23, wherein the lysis agent comprises a detergent.

25. The method according to claim 1, wherein the BoNT samples are selected from BoNT/A, BoNT/E, and BoNT/C.
